SEQUENCE LISTING (42 sequences)

SEQ ID NO: 1 - protein, 117 aa, synthetic construct
  REGION 1..117: note = Synthetic
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSS

SEQ ID NO: 2 - protein, 108 aa, synthetic construct
  REGION 1..108: note = Synthetic
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIK

SEQ ID NO: 3 - protein, 124 aa, synthetic construct
  REGION 1..124: note = Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 4 - protein, 124 aa, synthetic construct
  REGION 1..124: note = Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKDGSGYGKFYYYGMDVWGQGTTVTVSS

SEQ ID NO: 5 - protein, 124 aa, synthetic construct
  REGION 1..124: note = Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKDGSGYGKFYHYGLDVWGQGTTVTVSS

SEQ ID NO: 6 - protein, 124 aa, synthetic construct
  REGION 1..124: note = Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYYYGLDVWGQGTTVTVSS

SEQ ID NO: 7 - protein, 124 aa, synthetic construct
  REGION 1..124: note = Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 8 - protein, 8 aa, synthetic construct
  REGION 1..8: note = Synthetic
GFTFSNYY

SEQ ID NO: 9 - protein, 8 aa, synthetic construct
  REGION 1..8: note = Synthetic
ISGRGSTI

SEQ ID NO: 10 - protein, 10 aa, synthetic construct
  REGION 1..10: note = Synthetic
VKDRGGYSPY

SEQ ID NO: 11 - protein, 6 aa, synthetic construct
  REGION 1..6: note = Synthetic
QSISTY

SEQ ID NO: 12 - skipped

SEQ ID NO: 13 - protein, 10 aa, synthetic construct
  REGION 1..10: note = Synthetic
QQSYSTPPIT

SEQ ID NO: 14 - protein, 8 aa, synthetic construct
  REGION 1..8: note = Synthetic
GFTFDDYS

SEQ ID NO: 15 - protein, 8 aa, synthetic construct
  REGION 1..8: note = Synthetic
ISWNSGSK

SEQ ID NO: 16 - protein, 17 aa, synthetic construct
  REGION 1..17: note = Synthetic
AKYGSGYGKFYHYGLDV

SEQ ID NO: 17 - protein, 8 aa, synthetic construct
  REGION 1..8: note = Synthetic
GFTFDDYS

SEQ ID NO: 18 - protein, 8 aa, synthetic construct
  REGION 1..8: note = Synthetic
ISWNSGSI

SEQ ID NO: 19 - protein, 17 aa, synthetic construct
  REGION 1..17: note = Synthetic
AKDGSGYGKFYYYGMDV

SEQ ID NO: 20 - protein, 8 aa, synthetic construct
  REGION 1..8: note = Synthetic
GFTFDDYS

SEQ ID NO: 21 - protein, 8 aa, synthetic construct
  REGION 1..8: note = Synthetic
ISWNSGSK

SEQ ID NO: 22 - protein, 17 aa, synthetic construct
  REGION 1..17: note = Synthetic
AKDGSGYGKFYHYGLDV

SEQ ID NO: 23 - protein, 8 aa, synthetic construct
  REGION 1..8: note = Synthetic
GFTFDDYS

SEQ ID NO: 24 - protein, 8 aa, synthetic construct
  REGION 1..8: note = Synthetic
ISWNSGSK

SEQ ID NO: 25 - protein, 17 aa, synthetic construct
  REGION 1..17: note = Synthetic
AKYGSGYGKFYYYGLDV

SEQ ID NO: 26 - protein, 8 aa, synthetic construct
  REGION 1..8: note = Synthetic
GFTFDDYS

SEQ ID NO: 27 - protein, 8 aa, synthetic construct
  REGION 1..8: note = Synthetic
ISWNSGSI

SEQ ID NO: 28 - protein, 17 aa, synthetic construct
  REGION 1..17: note = Synthetic
AKYGSGYGKFYYYGMDV

SEQ ID NO: 29 - protein, 443 aa, synthetic construct
  REGION 1..443: note = Synthetic
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 215 aa, synthetic construct
  REGION 1..215: note = Synthetic
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 450 aa, synthetic construct
  REGION 1..450: note = Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 32 - protein, 450 aa, synthetic construct
  REGION 1..450: note = Synthetic
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 33 - protein, 116 aa, synthetic construct
  REGION 1..116: note = Synthetic
EVQLVESGGGLVQPGGSLRLSCAASGFTFSNYEMSWVRQAPGKGLEWVSSIRTSGTTKYYADSMKGRFTISRDNAKNSLYLQMNSLRAEDTAVYYCAGGGTFLHYWGQGTLVTVSS

SEQ ID NO: 34 - protein, 108 aa, synthetic construct
  REGION 1..108: note = Synthetic
DIQMTQSPSSVSASVGDRVTITCRASQGIASYLAWYQQKPGKAPKLLIYAASSLQTGVPSRFSGSGYGTDFTLTISSLQPEDFATYYCQQAKSFPMYTFGQGTKLEIK

SEQ ID NO: 35 - protein, 8 aa, synthetic construct
  REGION 1..8: note = Synthetic
GFTFSNYE

SEQ ID NO: 36 - protein, 8 aa, synthetic construct
  REGION 1..8: note = Synthetic
IRTSGTTK

SEQ ID NO: 37 - protein, 9 aa, synthetic construct
  REGION 1..9: note = Synthetic
AGGGTFLHY

SEQ ID NO: 38 - protein, 6 aa, synthetic construct
  REGION 1..6: note = Synthetic
QGIASY

SEQ ID NO: 39 - skipped

SEQ ID NO: 40 - protein, 10 aa, synthetic construct
  REGION 1..10: note = Synthetic
QQAKSFPMYT

SEQ ID NO: 41 - protein, 446 aa, synthetic construct
  REGION 1..446: note = Synthetic
EVQLVESGGGLVQPGGSLRLSCAASGFTFSNYEMSWVRQAPGKGLEWVSSIRTSGTTKYYADSMKGRFTISRDNAKNSLYLQMNSLRAEDTAVYYCAGGGTFLHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 215 aa, synthetic construct
  REGION 1..215: note = Synthetic
DIQMTQSPSSVSASVGDRVTITCRASQGIASYLAWYQQKPGKAPKLLIYAASSLQTGVPSRFSGSGYGTDFTLTISSLQPEDFATYYCQQAKSFPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0235089 A1
(43) Pub. Date: Jul. 27, 2023

(54) Methods for Treating Cancer with Bispecific Anti-CD3 x MUC16 Antibodies and Anti-CTLA-4 Antibodies

(71) Applicant: Regeneron Pharmaceuticals, Inc., Tarrytown, NY (US)

(72) Inventors: Ella Ioffe, Bronx, NY (US); Elena Burova, Mount Kisco, NY (US)

(21) Appl. No.: 17/993,654

(22) Filed: Nov. 23, 2022

Related U.S. Application Data
(60) Provisional application No. 63/282,811, filed on Nov. 24, 2021.

(51) Int. Cl. C07K 16/46; C07K 16/28; A61P 35/00

(52) U.S. Cl. CPC C07K 16/468; C07K 16/2818; A61P 35/00; C07K 2317/565; C07K 2317/31

(57) ABSTRACT

The present invention provides methods for treating, reducing the severity, or inhibiting the growth of cancer (e.g., ovarian cancer or pancreatic cancer). The methods of the present invention comprise administering to a subject in need thereof a therapeutically effective amount of an antibody or antigen-binding fragment thereof that specifically binds to immunomodulatory receptor cytotoxic T-lymphocyte-associated protein 4 (CTLA-4) in combination with a therapeutically effective amount of a bispecific antibody that specifically binds Mucin 16 (MUC16) and CD3.

### CROSS-REFERENCE TO RELATED APPLICATION

This application claims the benefit under 35 USC § 119(e) of U.S. Provisional Application No. 63/282,811, filed Nov. 24, 2021, which is incorporated herein by reference in its entirety for all purposes.

### REFERENCE TO A SEQUENCE LISTING

This application incorporates by reference a computer readable Sequence Listing in ST.26 XML format, titled 11047US01_Sequence, created on Nov. 23, 2022 and containing 54,045 bytes.

### FIELD OF THE INVENTION

The present invention relates to methods for treating cancer comprising administering to a subject in need thereof a therapeutically effective amount of an antibody that specifically binds to the immunomodulatory receptor cytotoxic T-lymphocyte-associated protein 4 (CTLA-4) in combination with a bispecific antibody that binds to mucin 16 (MUC16) and CD3.

### BACKGROUND

Mucin 16 (MUC16), also known as cancer antigen 125, carcinoma antigen 125, carbohydrate antigen 125, or CA-125, is a single transmembrane domain highly glycosylated integral membrane glycoprotein that is highly expressed in ovarian cancer. MUC16 consists of three major domains: an extracellular N-terminal domain, a large tandem repeat domain interspersed with sea urchin sperm, enterokinase, and agrin (SEA) domains, and a carboxyl terminal domain that comprises a segment of the transmembrane region and a short cytoplasmic tail. Proteolytic cleavage results in shedding of the extracellular portion of MUC16 into the bloodstream. MUC16 is overexpressed in cancers including ovarian cancer, breast cancer, pancreatic cancer, non-small-cell lung cancer, intrahepatic cholangiocarcinoma-mass forming type, adenocarcinoma of the uterine cervix, and adenocarcinoma of the gastric tract, and in diseases and conditions including inflammatory bowel disease, liver cirrhosis, cardiac failure, peritoneal infection, and abdominal surgery. (Haridas, D. et al., 2014, FASEB J., 28:4183-4199). Expression on cancer cells is shown to protect tumor cells from the immune system. (Felder, M. et al., 2014, Molecular Cancer, 13:129) Methods for treating ovarian cancer using antibodies to MUC16 have been investigated. Oregovomab and abgovomab are anti-MUC16 antibodies which have had limited success. (Felder, supra, Das, S. and Batra, S. K. 2015, Cancer Res. 75:4660-4674.)

CD3 is a homodimeric or heterodimeric antigen expressed on T cells in association with the T cell receptor complex (TCR) and is required for T cell activation. Functional CD3 is formed from the dimeric association of two of four different chains: epsilon, zeta, delta and gamma. The CD3 dimeric arrangements include gamma/epsilon, delta/epsilon and zeta/zeta. Antibodies against CD3 have been shown to cluster CD3 on T cells, thereby causing T cell activation in a manner similar to the engagement of the TCR by peptide-loaded MHC molecules. Thus, anti-CD3 antibodies have been proposed for therapeutic purposes involving the activation of T cells. In addition, bispecific antibodies that are capable of binding CD3 and a target antigen have been proposed for therapeutic uses involving targeting T cell immune responses to tissues and cells expressing the target antigen.

Cytotoxic T-lymphocyte-associated protein 4 (CTLA-4; also known as CD152) is a type I transmembrane T cell inhibitory checkpoint receptor expressed on conventional and regulatory T cells. CTLA-4 negatively regulates T cell activation by outcompeting the stimulatory receptor CD28 from binding to its natural ligands, B7-1 (CD80) and B7-2 (CD86). Initial T-cell activation is achieved by stimulating T-cell receptors (TCR) that recognize specific peptides presented by major histocompatibility complex class I or II (MHCI or MHCII) proteins on antigen-presenting cells (APC) (Goldrath et al. 1999, Nature 402: 255-262). An activated TCR in turn initiates a cascade of signaling events, which can be monitored by expression of transfected reporter genes, driven by promoters regulating the expression of various transcription factors such as activator-protein 1 (AP-1), Nuclear Factor of Activated T-cells (NFAT) or Nuclear factor kappa-light-chain-enhancer of activated B cells (NFκB). The T-cell response is then further refined via engagement of co-stimulatory or co-inhibitory receptors expressed either constitutively or inducibly on T-cells such as CD28, CTLA-4 (Cytotoxic T-Lymphocyte-Associated Protein 4), PD-1 (Programmed Cell Death Protein 1), LAG-3 (Lymphocyte-Activation Gene 3) or other molecules (Sharpe et al. 2002, Nat. Rev. Immunol. 2: 116-126).

Programmed death-1 (PD-1) receptor signaling in the tumor microenvironment plays a key role in allowing tumor cells to escape immune surveillance by the host immune system. Blockade of the PD-1 signaling pathway has demonstrated clinical activity in patients with multiple tumor types, and antibody therapeutics that block PD-1 (e.g., nivolumab and pembrolizumab) have been approved for the treatment of metastatic melanoma and metastatic squamous non-small cell lung cancer. Recent data has demonstrated the clinical activity of PD-1 blockade in patients with aggressive NHL and Hodgkin's lymphoma (Lesokhin, et al. 2014, Abstract 291, 56th ASH Annual Meeting and Exposition, San Francisco, Calif.; Ansell et al. 2015, N. Engl. J. Med. 372(4):311-9).

Ovarian cancer is the most lethal of the gynecologic malignancies; although the estimated number of new cases of ovarian cancer among American women are much lower than certain other cancers, the death-to-incidence ratio for ovarian cancer is considerably higher (Siegal et al., CA Cancer J Clin 66:7-30, 2016). Ovarian cancer is frequently diagnosed at an advanced stage, which contributes to its lethality. The current standard of care for ovarian cancer is surgery followed by chemotherapy, namely a combination of platinum agents and taxanes. Whilst the majority of patients respond to initial treatment, most experience a recurrence of the disease, resulting in a cycle of repeated surgeries and additional rounds of chemotherapy. Although recurrent ovarian cancers may respond to further treatment, virtually all of them will ultimately become resistant to currently available therapies. Despite recent advances in therapy such as PARP inhibitors for patients carrying BRCA or other homologous recombination deficiency (HRD) mutations, advanced ovarian cancer remains a disease of high unmet need.

Evidence suggests that ovarian cancer may be amenable to some forms of immunotherapy (Kandalaft et al., J. Clin. Oncol., 29:925-933, 2011). For example, ovarian cancer patients whose tumors were positive for intraepithelial CD8⁺ T lymphocyte infiltration had significantly better overall and progression-free survival than patients without intraepithelial CD8⁺ T lymphocyte infiltration (Hamanishi et al., PNAS, 104:3360-65, 2007; and Zhang et al., N. Engl. J. Med., 348:203-213, 2003). Moreover, some patients have shown spontaneous immune response to their tumors, demonstrated by detection of tumor-reactive T cells and antibodies in the blood, tumor or ascites of patients with advanced disease (Schliengar et al., Clin Cancer Res, 9:1517-1527, 2003).

An unresolved question is whether anti-tumor immune responses induced by anti-CTLA-4 and anti-PD-1 antibodies are mediated through distinct, non-redundant mechanisms. Many studies have demonstrated that CTLA-4 and PD-1 attenuate T cell activation through distinct mechanisms (Pardoll, Nat. Rev. Cancer, 12:252-264, 2012). CTLA-4 is upregulated immediately following TCR ligation and outcompetes CD28 for B7 ligand binding, thus attenuating positive costimulation by CD28 (Krummel and Allison, J. Exp. Med., 182:459-465, 1995; Walunas et al., Immunity, 1:405-413, 1994). PD-1 is induced later during T cell activation and, upon engagement with PD-L1 or PD-L2, attenuates TCR signaling via recruitment of tyrosine phosphatases. In addition to utilizing distinct molecular mechanisms of action, CTLA-4 and PD-1 attenuate T cell activity through mechanisms that are separated spatially and temporally. Whereas CTLA-4 primarily attenuates T cell activation in the priming phase through cell intrinsic and extrinsic mechanisms, PD-1 primarily attenuates T cell activity in peripheral tissues through cell intrinsic mechanisms (Pardoll, Id.; Walker and Sansom, Nat Rev Immunol., 11(12):852-853, 2011). This distinction is highlighted by the fact that the cellular sources of the ligands of PD-1 and CTLA-4 are different and serve different physiological functions (Wei, Cell, 170(6):1120-1133, 2017). Blockade of the PD-1 signaling pathway has demonstrated some clinical activity in patients with multiple tumor types. Anti-PD-1 antibody treatment is the focus of clinical combination treatment with many different modalities, including bispecific antibodies for the treatment of ovarian cancer.

In view of the high unmet need for effective therapies for ovarian cancer, it may be useful, as shown herein, to combine treatment with an agent to augment T-cell function (e.g., a CTLA-4 inhibitor such as an anti-CTLA-4 antibody) along with an agent against a target antigen (a bispecific anti-MUC16/anti-CD3 antibody).

### BRIEF SUMMARY OF THE INVENTION

According to certain embodiments, the present invention provides methods for treating, ameliorating at least one symptom or indication, or inhibiting the growth of a MUC16-expressing cancer in a subject. The methods according to this aspect of the invention comprise administering a therapeutically effective amount of an antibody or antigen-binding fragment thereof that specifically binds to cytotoxic T-lymphocyte-associated protein 4 (CTLA-4) in combination with a therapeutically effective amount of a bispecific antibody that specifically binds to MUC16 and CD3 to a subject in need thereof.

In certain embodiments of the present invention, methods are provided for treating, ameliorating at least one symptom or indication, or inhibiting the growth of MUC16-expressing cancer in a subject. In certain embodiments of the present invention, methods are provided for delaying the growth of a tumor or preventing tumor recurrence. The methods, according to this and other aspects of the invention, comprise sequentially administering one or more doses of a therapeutically effective amount of an antibody or antigen-binding fragment thereof that specifically binds to CTLA-4 in combination with one or more doses of a therapeutically effective amount of a bispecific antibody that specifically binds to MUC16 and CD3 to a subject in need thereof.

In one aspect, the present invention provides a method of treating or inhibiting the growth of a MUC16-expressing tumor comprising administering to a subject in need thereof (a) a therapeutically effective amount of an antibody or antigen-binding fragment thereof that specifically binds cytotoxic T-lymphocyte-associated protein 4 (CTLA-4); and (b) a therapeutically effective amount of a bispecific antibody comprising a first antigen-binding arm that specifically binds MUC16 and a second antigen-binding arm that specifically binds CD3. In some cases, the anti-CTLA-4 antibody is administered prior to, concurrent with or after the bispecific antibody. In some cases, the anti-CTLA-4 antibody is administered prior to the bispecific antibody. In some cases, the anti-CTLA-4 antibody is administered after the bispecific antibody. In some cases, one or more doses of the anti-CTLA-4 antibody are administered in combination with one or more doses of the bispecific antibody. In some cases, the anti-CTLA-4 antibody is administered at a dose of between 0.1 mg/kg and 20 mg/kg of the subject's body weight. In some cases, the anti-CTLA-4 antibody is administered at a dose of from 10 mg to 1500 mg (e.g., from 20 mg to 250 mg). In some cases, the bispecific antibody is administered at a dose of between 0.1 mg/kg and 20 mg/kg of the subject's body weight. In some cases, the bispecific antibody is administered at a dose of from 10 mg to 1500 mg (e.g., from 30 mg to 900 mg). In some cases, each subsequent dose of the anti-CTLA-4 antibody is administered 0.5-12 weeks after the immediately preceding dose. In some cases, each subsequent dose of the bispecific antibody is administered 0.5-12 weeks after the immediately preceding dose. In various embodiments, the antibodies are administered intravenously, subcutaneously, or intraperitoneally.

In some embodiments, the tumor is selected from an ovarian cancer, ovarian serous carcinoma, a breast cancer, invasive lobular breast carcinoma, a pancreatic cancer, pancreatic ductal adenocarcinoma, a bladder cancer, or non-small cell lung cancer. In some embodiments, the tumor comprises an ovarian cancer. In some embodiments, the tumor comprises ovarian serous carcinoma. In some embodiments, the subject is resistant to or inadequately responsive to, or relapsed after, prior therapy.

In some cases, the method further comprises administering to the subject a third therapeutic agent or therapy. In some embodiments, the third therapeutic agent or therapy is selected from the group consisting of radiation, surgery, a chemotherapeutic agent, a cancer vaccine, a PD-1 inhibitor, a PD-L1 inhibitor, a LAG-3 inhibitor, a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGF.beta.) inhibitor, an epidermal growth factor receptor (EGFR) inhibitor, an antibody to a tumor-specific antigen, Bacillus Calmette-Guerin vaccine, granulocyte-macrophage colony-stimulating factor, a cytotoxin, an interleukin 6 receptor (IL-6R) inhibitor, an interleukin 4 receptor (IL-4R) inhibitor, an IL-10 inhibitor, IL-2, IL-7, IL-21, IL-15, an antibody-drug conjugate, an anti-inflammatory drug, and a dietary supplement.

In some embodiments, the anti-CTLA4 antibody or antigen-binding fragment thereof is selected from ipilimumab and REGN4659. In some embodiments, the anti-CTLA-4 antibody or antigen-binding fragment thereof comprises the heavy chain complementarity determining regions (HCDR1, HCDR2 and HCDR3) of a heavy chain variable region (HCVR) comprising the amino acid sequence of SEQ ID NO: 33, and three light chain complementarity determining regions (LCDR1, LCDR2 and LCDR3) of a light chain variable region (LCVR) comprising the amino acid sequence of SEQ ID NO: 34. In some cases, HCDR1 comprises the amino acid sequence of SEQ ID NO: 35; HCDR2 comprises the amino acid sequence of SEQ ID NO: 36; HCDR3 comprises the amino acid sequence of SEQ ID NO: 37; LCDR1 comprises the amino acid sequence of SEQ ID NO: 38; LCDR2 comprises the amino acid sequence of SEQ ID NO: 39; and LCDR3 comprises the amino acid sequence of SEQ ID NO: 40. In some cases, the anti-CTLA4 antibody or antigen-binding fragment thereof comprises a HCVR comprising the amino acid sequence of SEQ ID NO: 33, and a LCVR comprising the amino acid sequence of SEQ ID NO: 34. In some embodiments, the anti-CTLA-4 antibody comprises a heavy chain comprising the amino acid sequence of SEQ ID NO: 41, and a light chain comprising the amino acid sequence of SEQ ID NO: 42.

In some embodiments, the first antigen-binding arm of the bispecific antibody comprises three heavy chain CDRs (A-HCDR1, A-HCDR2 and A-HCDR3) of a heavy chain variable region (A-HCVR) comprising the amino acid sequence of SEQ ID NO: 1 and three light chain CDRs (A-LCDR1, A-LCDR2 and A-LCDR3) of a light chain variable region (A-LCVR) comprising the amino acid sequence of SEQ ID NO: 2. In some cases, A-HCDR1 comprises the amino acid sequence of SEQ ID NO: 8; A-HCDR2 comprises the amino acid sequence of SEQ ID NO: 9; A-HCDR3 comprises the amino acid sequence of SEQ ID NO: 10; A-LCDR1 comprises the amino acid sequence of SEQ ID NO: 11; A-LCDR2 comprises the amino acid sequence of SEQ ID NO: 12; and A-LCDR3 comprises the amino acid sequence of SEQ ID NO: 13. In some cases, the bispecific antibody comprises a A-HCVR comprising the amino acid sequence of SEQ ID NO:1 and a A-LCVR comprising the amino acid sequence of SEQ ID NO:2.

In some embodiments, the second antigen-binding arm of the bispecific antibody comprises three heavy chain CDRs (B-HCDR1, B-HCDR2 and B-HCDR3) of a heavy chain variable region (B-HCVR) comprising an amino acid sequence selected from the group consisting of SEQ ID NOs: 3, 4, 5, 6 and 7, and three light chain CDRs (B-LCDR1, B-LCDR2 and B-LCDR3) of a light chain variable region (B-LCVR) comprising the amino acid sequence of SEQ ID NO: 2. In some cases, B-HCDR1, B-HCDR2 and B-HCDR3 comprise, respectively, the amino acid sequences selected from the group consisting of SEQ ID NOs: 14-15-16, 17-18-19, 20-21-22, 23-24-25, and 26-27-28; and B-LCDR1, B-LCDR2 and B-LCDR3 comprise, respectively, the amino acid sequences of SEQ ID NOs: 11-12-13. In some cases, the second antigen-binding arm comprises a B-HCVR comprising an amino acid sequence selected from the group consisting of SEQ ID NOs: 3, 4, 5, 6 and 7, and a B-LCVR comprising the amino acid sequence of SEQ ID NO: 2.

In some embodiments, the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3, and three light chain CDRs (B-LCDR1, B-LCDR2 and B-LCDR3) of a light chain variable region (B-LCVR) comprising the amino acid sequence of SEQ ID NO: 2.

In some embodiments, the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 4, and three light chain CDRs (B-LCDR1, B-LCDR2 and B-LCDR3) of a light chain variable region (B-LCVR) comprising the amino acid sequence of SEQ ID NO: 2.

In some embodiments, the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 5, and three light chain CDRs (B-LCDR1, B-LCDR2 and B-LCDR3) of a light chain variable region (B-LCVR) comprising the amino acid sequence of SEQ ID NO: 2.

In some embodiments, the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 6, and three light chain CDRs (B-LCDR1, B-LCDR2 and B-LCDR3) of a light chain variable region (B-LCVR) comprising the amino acid sequence of SEQ ID NO: 2.

In some embodiments, the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 7, and three light chain CDRs (B-LCDR1, B-LCDR2 and B-LCDR3) of a light chain variable region (B-LCVR) comprising the amino acid sequence of SEQ ID NO: 2.

In some embodiments, the anti-CTLA-4 antibody, the bispecific antibody, or both, comprise a human IgG1 or IgG4 heavy chain constant region.

In another aspect, the present invention provides a method of treating or inhibiting the growth of a MUC16-expressing tumor comprising administering to a subject in need thereof (a) a therapeutically effective amount of an antibody or antigen-binding fragment thereof that specifically binds cytotoxic T-lymphocyte-associated protein 4 (CTLA-4); and (b) a therapeutically effective amount of a bispecific antibody comprising a first antigen-binding arm that specifically binds MUC16 and a second antigen-binding arm that specifically binds CD3, wherein: (a) the anti-CTLA-4 antibody or antigen-binding fragment thereof comprises the heavy chain complementarity determining regions (HCDR1, HCDR2 and HCDR3) of a heavy chain variable region (HCVR) comprising the amino acid sequence of SEQ ID NO: 33, and three light chain complementarity determining regions (LCDR1, LCDR2 and LCDR3) of a light chain variable region (LCVR) comprising the amino acid sequence of SEQ ID NO: 34; (b) the first antigen-binding arm of the bispecific antibody comprises three heavy chain CDRs (A-HCDR1, A-HCDR2 and A-HCDR3) of a heavy chain variable region (A-HCVR) comprising the amino acid sequence of SEQ ID NO: 1 and three light chain CDRs (A-LCDR1, A-LCDR2 and A-LCDR3) of a light chain variable region (A-LCVR) comprising the amino acid sequence of SEQ ID NO: 2; and (c) the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3, and three light chain CDRs (B-LCDR1, B-LCDR2 and B-LCDR3) of a light chain variable region (B-LCVR) comprising the amino acid sequence of SEQ ID NO: 2.

In some embodiments of the method, the anti-CTLA-4 antibody and the bispecific antibody, respectively include the following: (a) HCDR1 comprises the amino acid sequence of SEQ ID NO: 35; HCDR2 comprises the amino acid sequence of SEQ ID NO: 36; HCDR3 comprises the amino acid sequence of SEQ ID NO: 37; LCDR1 comprises the amino acid sequence of SEQ ID NO: 38; LCDR2 comprises the amino acid sequence of SEQ ID NO: 39; and LCDR3 comprises the amino acid sequence of SEQ ID NO: 40; (b) A-HCDR1 comprises the amino acid sequence of SEQ ID NO: 8; A-HCDR2 comprises the amino acid sequence of SEQ ID NO: 9; A-HCDR3 comprises the amino acid sequence of SEQ ID NO: 10; A-LCDR1 comprises the amino acid sequence of SEQ ID NO: 11; A-LCDR2 comprises the amino acid sequence of SEQ ID NO: 12; and A-LCDR3 comprises the amino acid sequence of SEQ ID NO: 13; and (c) B-HCDR1 comprises the amino acid sequence of SEQ ID NO: 14; B-HCDR2 comprises the amino acid sequence of SEQ ID NO: 15; B-HCDR3 comprises the amino acid sequence of SEQ ID NO: 16; B-LCDR1 comprises the amino acid sequence of SEQ ID NO: 11; B-LCDR2 comprises the amino acid sequence of SEQ ID NO: 12; and B-LCDR3 comprises the amino acid sequence of SEQ ID NO: 13.

In some embodiments of the method, that anti-CTLA-4 antibody and the bispecific antibody, respectively, include the following: (a) the HCVR comprises the amino acid sequence of SEQ ID NO: 33, and the LCVR comprises the amino acid sequence of SEQ ID NO: 34; (b) the A-HCVR comprises the amino acid sequence of SEQ ID NO:1 and the A-LCVR comprises the amino acid sequence of SEQ ID NO:2; and (c) the B-HCVR comprises the amino acid sequence of SEQ ID NO: 3, and the B-LCVR comprises the amino acid sequence of SEQ ID NO: 2.

In some embodiments of the method, the anti-CTLA-4 antibody comprises a heavy chain comprising the amino acid sequence of SEQ ID NO: 41, and a light chain comprising the amino acid sequence of SEQ ID NO: 42; the first antigen binding arm of the bispecific antibody comprises a heavy chain comprising the amino acid sequence of SEQ ID NO: 29, and a light chain comprising the amino acid sequence of SEQ ID NO: 30; and the second antigen binding arm of the bispecific antibody comprises a heavy chain comprising the amino acid sequence of SEQ ID NO: 31, and a light chain comprising the amino acid sequence of SEQ ID NO: 30.

In the various embodiments of the methods discussed above or herein, administration of the anti-CTLA4 antibody or antigen-binding fragment thereof and/or the bispecific antibody does not cause a significant increase in serum cytokine levels relative to a control (e.g., isotype control). Thus, administration of the combination of the anti-CTLA4 antibody (or fragment thereof) and the bispecific antibody to treat a MUC16-expressing cancer can be undertaken without inducing cytokine release syndrome in the subject administered the combination therapy. In some cases, administration of the anti-CTLA4 antibody or antigen-binding fragment thereof and the bispecific antibody results in an increase in serum cytokine levels in the subject of no more than 5% relative to an isotype control at four hours post-administration. In some cases, administration of the anti-CTLA4 antibody or antigen-binding fragment thereof and the bispecific antibody results in an increase in serum cytokine levels in the subject of no more than 4%, no more than 3%, no more than 2%, or no more than 1%, relative to an isotype control at four hours post-administration.

In some embodiments, the antibodies discussed herein are used in the manufacture of a medicament for use in any of the methods discussed above or herein. In some embodiments, the antibodies discussed herein are for use in medicine or for use in the treatment of cancer as discussed above or herein. For example, the present disclosure includes:

(A) Use of a bispecific antibody comprising a first antigen-binding arm that specifically binds MUC16 and a second antigen-binding arm that specifically binds CD3 in the manufacture of a medicament for treating or inhibiting the growth of a MUC16-expressing tumor in a subject in need thereof in combination with an antibody or antigen-binding fragment thereof that specifically binds cytotoxic T-lymphocyte-associated protein 4 (CTLA-4);

(B) Use of an antibody or antigen-binding fragment thereof that specifically binds cytotoxic T-lymphocyte-associated protein 4 (CTLA-4) in the manufacture of a medicament for treating or inhibiting the growth of a MUC16-expressing tumor in a subject in need thereof in combination with a bispecific antibody comprising a first antigen-binding arm that specifically binds MUC16 and a second antigen-binding arm that specifically binds CD3;

(C) Use of a bispecific antibody comprising a first antigen-binding arm that specifically binds MUC16 and a second antigen-binding arm that specifically binds CD3 in the manufacture of a medicament for treating or inhibiting the growth of a MUC16-expressing tumor in a subject in need thereof in combination with an antibody or antigen-binding fragment thereof that specifically binds cytotoxic T-lymphocyte-associated protein 4 (CTLA-4), wherein: (i) the anti-CTLA-4 antibody or antigen-binding fragment thereof comprises the heavy chain complementarity determining regions (HCDR1, HCDR2 and HCDR3) of a heavy chain variable region (HCVR) comprising the amino acid sequence of SEQ ID NO: 33, and three light chain complementarity determining regions (LCDR1, LCDR2 and LCDR3) of a light chain variable region (LCVR) comprising the amino acid sequence of SEQ ID NO: 34; (ii) the first antigen-binding arm of the bispecific antibody comprises three heavy chain CDRs (A-HCDR1, A-HCDR2 and A-HCDR3) of a heavy chain variable region (A-HCVR) comprising the amino acid sequence of SEQ ID NO: 1 and three light chain CDRs (A-LCDR1, A-LCDR2 and A-LCDR3) of a light chain variable region (A-LCVR) comprising the amino acid sequence of SEQ ID NO: 2; and (iii) the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3, and three light chain CDRs (B-LCDR1, B-LCDR2 and B-LCDR3) of a light chain variable region (B-LCVR) comprising the amino acid sequence of SEQ ID NO: 2;

(D) Use of an antibody or antigen-binding fragment thereof that specifically binds cytotoxic T-lymphocyte-associated protein 4 (CTLA-4) in the manufacture of a medicament for treating or inhibiting the growth of a MUC16-expressing tumor in a subject in need thereof in combination with a bispecific antibody comprising a first antigen-binding arm that specifically binds MUC16 and a second antigen-binding arm that specifically binds CD3, wherein: (i) the anti-CTLA-4 antibody or antigen-binding fragment thereof comprises the heavy chain complementarity determining regions (HCDR1, HCDR2 and HCDR3) of a heavy chain variable region (HCVR) comprising the amino acid sequence of SEQ ID NO: 33, and three light chain complementarity determining regions (LCDR1, LCDR2 and LCDR3) of a light chain variable region (LCVR) comprising the amino acid sequence of SEQ ID NO: 34; (ii) the first antigen-binding arm of the bispecific antibody comprises three heavy chain CDRs (A-HCDR1, A-HCDR2 and A-HCDR3) of a heavy chain variable region (A-HCVR) comprising the amino acid sequence of SEQ ID NO: 1 and three light chain CDRs (A-LCDR1, A-LCDR2 and A-LCDR3) of a light chain variable region (A-LCVR) comprising the amino acid sequence of SEQ ID NO: 2; and (iii) the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3, and three light chain CDRs (B-LCDR1, B-LCDR2 and B-LCDR3) of a light chain variable region (B-LCVR) comprising the amino acid sequence of SEQ ID NO: 2;

(E) A bispecific antibody comprising a first antigen-binding arm that specifically binds MUC16 and a second antigen-binding arm that specifically binds CD3 for use in treating or inhibiting the growth of a MUC16-expressing tumor in a subject in need thereof in combination with an antibody or antigen-binding fragment thereof that specifically binds cytotoxic T-lymphocyte-associated protein 4 (CTLA-4);

(F) An antibody or antigen-binding fragment thereof that specifically binds cytotoxic T-lymphocyte-associated protein 4 (CTLA-4) for use in treating or inhibiting the growth of a MUC16-expressing tumor in a subject in need thereof in combination with a bispecific antibody comprising a first antigen-binding arm that specifically binds MUC16 and a second antigen-binding arm that specifically binds CD3;

(G) A bispecific antibody comprising a first antigen-binding arm that specifically binds MUC16 and a second antigen-binding arm that specifically binds CD3 for use in treating or inhibiting the growth of a MUC16-expressing tumor in a subject in need thereof in combination with an antibody or antigen-binding fragment thereof that specifically binds cytotoxic T-lymphocyte-associated protein 4 (CTLA-4), wherein: (i) the anti-CTLA-4 antibody or antigen-binding fragment thereof comprises the heavy chain complementarity determining regions (HCDR1, HCDR2 and HCDR3) of a heavy chain variable region (HCVR) comprising the amino acid sequence of SEQ ID NO: 33, and three light chain complementarity determining regions (LCDR1, LCDR2 and LCDR3) of a light chain variable region (LCVR) comprising the amino acid sequence of SEQ ID NO: 34; (ii) the first antigen-binding arm of the bispecific antibody comprises three heavy chain CDRs (A-HCDR1, A-HCDR2 and A-HCDR3) of a heavy chain variable region (A-HCVR) comprising the amino acid sequence of SEQ ID NO: 1 and three light chain CDRs (A-LCDR1, A-LCDR2 and A-LCDR3) of a light chain variable region (A-LCVR) comprising the amino acid sequence of SEQ ID NO: 2; and (iii) the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3, and three light chain CDRs (B-LCDR1, B-LCDR2 and B-LCDR3) of a light chain variable region (B-LCVR) comprising the amino acid sequence of SEQ ID NO: 2; and

(H) An antibody or antigen-binding fragment thereof that specifically binds cytotoxic T-lymphocyte-associated protein 4 (CTLA-4) for use in treating or inhibiting the growth of a MUC16-expressing tumor in a subject in need thereof in combination with a bispecific antibody comprising a first antigen-binding arm that specifically binds MUC16 and a second antigen-binding arm that specifically binds CD3, wherein: (i) the anti-CTLA-4 antibody or antigen-binding fragment thereof comprises the heavy chain complementarity determining regions (HCDR1, HCDR2 and HCDR3) of a heavy chain variable region (HCVR) comprising the amino acid sequence of SEQ ID NO: 33, and three light chain complementarity determining regions (LCDR1, LCDR2 and LCDR3) of a light chain variable region (LCVR) comprising the amino acid sequence of SEQ ID NO: 34; (ii) the first antigen-binding arm of the bispecific antibody comprises three heavy chain CDRs (A-HCDR1, A-HCDR2 and A-HCDR3) of a heavy chain variable region (A-HCVR) comprising the amino acid sequence of SEQ ID NO: 1 and three light chain CDRs (A-LCDR1, A-LCDR2 and A-LCDR3) of a light chain variable region (A-LCVR) comprising the amino acid sequence of SEQ ID NO: 2; and (iii) the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3, and three light chain CDRs (B-LCDR1, B-LCDR2 and B-LCDR3) of a light chain variable region (B-LCVR) comprising the amino acid sequence of SEQ ID NO: 2.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

Other embodiments of the present invention will become apparent from a review of the ensuing detailed description.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention. Any specific value discussed above or herein may be combined with another related value discussed above or herein to recite a range with the values representing the upper and lower ends of the range, and such ranges are encompassed within the scope of the present disclosure.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Methods for Treating or Inhibiting the Growth of Cancers

The present invention includes methods for treating, ameliorating or reducing the severity of at least one symptom or indication, or inhibiting the growth of a cancer (e.g., a MUC16-expressing cancer) in a subject. The methods according to this aspect of the invention comprise administering a therapeutically effective amount of an antibody or antigen-binding fragment thereof that specifically binds CTLA-4 in combination with a therapeutically effective amount of a bispecific antibody against MUC16 and CD3 to a subject in need thereof. As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, and/or to increase duration of survival of the subject.

As used herein, the expression “a subject in need thereof” means a human or non-human mammal that exhibits one or more symptoms or indications of cancer, and/or who has been diagnosed with cancer, including an ovarian cancer and who needs treatment for the same. In many embodiments, the term “subject” may be interchangeably used with the term “patient”. For example, a human subject may be diagnosed with a primary or a metastatic tumor and/or with one or more symptoms or indications including, but not limited to, enlarged lymph node(s), swollen abdomen, chest pain/pressure, unexplained weight loss, fever, night sweats, persistent fatigue, loss of appetite, enlargement of spleen, itching. The expression includes subjects with primary or established ovarian tumors. In specific embodiments, the expression includes human subjects that have and need treatment for ovarian cancer or another tumor expressing MUC16. In other specific embodiments, the expression includes subjects with MUC16+ tumors (e.g., a tumor with MUC16 expression as determined by flow cytometry). In certain embodiments, the expression “a subject in need thereof” includes patients with an ovarian cancer that is resistant to or refractory to or is inadequately controlled by prior therapy (e.g., treatment with a conventional anti-cancer agent). For example, the expression includes subjects who have been treated with chemotherapy, such as a platinum-based chemotherapeutic agent (e.g., cisplatin) or a taxol compound (e.g., docetaxel). The expression also includes subjects with an ovarian tumor for which conventional anti-cancer therapy is inadvisable, for example, due to toxic side effects. For example, the expression includes patients who have received one or more cycles of chemotherapy with toxic side effects. In certain embodiments, the expression “a subject in need thereof” includes patients with an ovarian tumor which has been treated but which has subsequently relapsed or metastasized. For example, patients with an ovarian tumor that may have received treatment with one or more anti-cancer agents leading to tumor regression; however, subsequently have relapsed with cancer resistant to the one or more anti-cancer agents (e.g., chemotherapy-resistant cancer) are treated with the methods of the present invention.

The expression “a subject in need thereof” also includes subjects who are at risk of developing ovarian cancer, e.g., persons with a family history of ovarian cancer, persons with a past history of infections associated with ovarian cancer, persons with mutations in the BRCA1/2 genes, or persons with an immune system compromised due to HIV infection or due to immunosuppressive medications.

In certain embodiments, the methods of the present invention are used in a subject with an ovarian cancer. The terms “tumor”, “cancer” and “malignancy” are interchangeably used herein. The term “ovarian cancer”, as used herein, refers to tumors of the ovary and fallopian tube, and includes serous cancer, endometrioid carcinoma, clear cell carcinoma, and mucinous carcinoma.

According to certain embodiments, the present invention includes methods for treating, or delaying or inhibiting the growth of a tumor. In certain embodiments, the present invention includes methods to promote tumor regression. In certain embodiments, the present invention includes methods to reduce tumor cell load or to reduce tumor burden. In certain embodiments, the present invention includes methods to prevent tumor recurrence. The methods, according to this aspect of the invention, comprise sequentially administering a therapeutically effective amount of an anti-CTLA-4 antibody in combination with a bispecific anti-MUC16/anti-CD3 antibody to a subject in need thereof, wherein each antibody is administered to the subject in multiple doses, e.g., as part of a specific therapeutic dosing regimen. For example, the therapeutic dosing regimen may comprise administering one or more doses of an anti-CTLA-4 antibody to the subject at a frequency of about once a day, once every two days, once every three days, once every four days, once every five days, once every six days, once a week, once every two weeks, once every three weeks, once every four weeks, once a month, once every two months, once every three months, once every four months, or less frequently. In certain embodiments, the one or more doses of anti-CTLA-4 antibody are administered in combination with one or more doses of a therapeutically effective amount of a bispecific anti-MUC16/anti-CD3 antibody, wherein the one or more doses of the bispecific antibody are administered to the subject at a frequency of about once a day, once every two days, once every three days, once every four days, once every five days, once every six days, once a week, once every two weeks, once every three weeks, once every four weeks, once a month, once every two months, once every three months, once every four months, or less frequently.

In certain embodiments, each dose of the anti-MUC16/anti-CD3 antibody is administered in more than 1 fractions, e.g., in 2-5 fractions (“split dosing”) within the given dosing period. The anti-MUC16/anti-CD3 bispecific antibody may be administered in split doses to reduce or eliminate the cytokine “spikes” induced in response to administration of the antibody. Cytokine spikes refer to the clinical symptoms of the cytokine release syndrome (“cytokine storm”) and infusion related reactions. In certain embodiments, the methods of the present invention comprise administering one or more doses of anti-CTLA-4 antibody in combination with one or more doses of a bispecific anti-MUC16/anti-CD3 antibody to a subject in need thereof, wherein a dose of the bispecific antibody is administered as split doses, or in more than 1 fractions, e.g., as 2 fractions, as 3 fractions, as 4 fractions or as 5 fractions within the given dosing period. In certain embodiments, a dose of the bispecific antibody is split into 2 or more fractions, wherein each fraction comprises an amount of the antibody equal to the other fractions. For example, a dose of anti-MUC16/anti-CD3 antibody comprising 1000 micrograms may be administered once a week, wherein the dose is administered in 2 fractions within the week, each fraction comprising 500 micrograms. In certain embodiments, a dose of the bispecific antibody is administered split into 2 or more fractions, wherein the fractions comprise unequal amounts of the antibody, e.g., more than or less than the first fraction. For example, a dose of anti-MUC16/anti-CD3 antibody comprising 1000 micrograms may be administered once a week, wherein the dose is administered in 2 fractions within the week, wherein the first fraction comprises 700 micrograms and the second fraction comprises 300 micrograms. As another example, a dose of anti-MUC16/anti-CD3 antibody comprising 1000 micrograms may be administered once in 2 weeks, wherein the dose is administered in 3 fractions within the 2-week period, wherein the first fraction comprises 400 micrograms, the second fraction comprises 300 micrograms and the third fraction comprises 300 micrograms.

In certain embodiments, the present invention includes methods to inhibit, retard or stop tumor metastasis or tumor infiltration into peripheral organs. The methods, according to this aspect, comprise administering a therapeutically effective amount of an anti-CTLA-4 antibody to a subject in need thereof in combination with a bispecific anti-MUC16/anti-CD3 antibody.

In specific embodiments, the present invention provides methods for increased anti-tumor efficacy or increased tumor inhibition. The methods, according to this aspect of the invention, comprise administering to a subject with an ovarian cancer a therapeutically effective amount of an anti-CTLA-4 antibody prior to administering a therapeutically effective amount of a bispecific anti-MUC16/anti-CD3 antibody, wherein the anti-CTLA-4 antibody may be administered about 1 day, more than 1 day, more than 2 days, more than 3 days, more than 4 days, more than 5 days, more than 6 days, more than 7 days, or more than 8 days prior to the bispecific antibody. In certain embodiments, the methods provide for increased tumor inhibition, e.g., by about 20%, more than 20%, more than 30%, more than 40% more than 50%, more than 60%, more than 70% or more than 80% as compared to a subject administered with the bispecific antibody prior to the anti-CTLA-4 antibody.

In certain embodiments, the methods of the present invention comprise administering a therapeutically effective amount of an anti-CTLA-4 antibody and a therapeutically effective amount of a bispecific anti-CD3×MUC16 antibody to a subject with an ovarian cancer. In specific embodiments, the ovarian cancer is serous cancer. In further embodiments, the ovarian cancer is indolent or aggressive. In certain embodiments, the subject is not responsive to prior therapy or has relapsed after prior therapy. In certain embodiments, the methods of the present invention further comprise administering an additional therapeutic agent to the subject.

In certain embodiments, the methods of the present invention comprise administering a therapeutically effective amount of a bispecific anti-MUC16/anti-CD3 antibody to a subject with a MUC16+ cancer. In specific embodiments, the cancer is an ovarian cancer. In further embodiments, the ovarian cancer is indolent or aggressive. In some embodiments, the cancer is a platinum-resistant ovarian cancer. In some embodiments, the cancer is a taxol-resistant ovarian cancer. In some embodiments, the cancer is fallopian tube cancer. In some embodiments, the cancer is primary peritoneal cancer, optionally in which the patient has elevated levels of serum CA-125. In specific embodiments, the cancer is pancreatic cancer (e.g., pancreatic adenocarcinoma). In certain embodiments, the subject is not responsive to prior therapy or has relapsed after prior therapy (e.g., chemotherapy).

In certain embodiments, the methods of the present invention comprise administering an anti-CTLA-4 antibody in combination with a bispecific anti-MUC16/anti-CD3 antibody to a subject in need thereof as a “first line” treatment (e.g., initial treatment). In other embodiments, an anti-CTLA-4 antibody in combination with a bispecific anti-MUC16/anti-CD3 antibody is administered as a “second line” treatment (e.g., after prior therapy). For example, an anti-CTLA-4 antibody in combination with a bispecific anti-MUC16/anti-CD3 antibody is administered as a “second line” treatment to a subject that has relapsed after prior therapy with, e.g., chemotherapy.

In certain embodiments, the methods of the present invention are used to treat a patient with a MRD-positive disease. Minimum residual disease (MRD) refers to small numbers of cancer cells that remain in the patient during or after treatment, wherein the patient may or may not show symptoms or signs of the disease. Such residual cancer cells, if not eliminated, frequently lead to relapse of the disease. The present invention includes methods to inhibit and/or eliminate residual cancer cells in a patient upon MRD testing. MRD may be assayed according to methods known in the art (e.g., MRD flow cytometry). The methods, according to this aspect of the invention, comprise administering an anti-CTLA-4 antibody in combination with a bispecific anti-MUC16/anti-CD3 antibody to a subject in need thereof.

The methods of the present invention, according to certain embodiments, comprise administering to a subject a therapeutically effective amount of each of an anti-CTLA-4 antibody and a bispecific anti-MUC16/anti-CD3 antibody in combination with a third therapeutic agent. The third therapeutic agent may be an agent selected from the group consisting of, e.g., radiation, chemotherapy, surgery, a cancer vaccine, a PD-1 inhibitor (e.g., an anti-PD-1 antibody), a PD-L1 inhibitor (e.g., an anti-PD-L1 antibody), a LAG3 inhibitor (e.g., an anti-LAG3 antibody), a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an Ang2 inhibitor, a transforming growth factor beta (TGF.beta.) inhibitor, an epidermal growth factor receptor (EGFR) inhibitor, an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin), granulocyte-macrophage colony-stimulating factor, a cytotoxin, a chemotherapeutic agent, an IL-6R inhibitor, an IL-4R inhibitor, an IL-10 inhibitor, a cytokine such as IL-2, IL-7, IL-21, and IL-15, an anti-inflammatory drug such as corticosteroids, and non-steroidal anti-inflammatory drugs, and a dietary supplement such as anti-oxidants. In certain embodiments, the antibodies may be administered in combination with therapy including a chemotherapeutic agent (e.g., paclitaxel, carboplatin, doxorubicin, cyclophosphamide, cisplatin, gemcitabine or docetaxel), radiation and surgery. As used herein, the phrase “in combination with” means that the antibodies are administered to the subject at the same time as, just before, or just after administration of the third therapeutic agent. In certain embodiments, the third therapeutic agent is administered as a co-formulation with the antibodies. In a related embodiment, the present invention includes methods comprising administering a therapeutically effective amount of an anti-CTLA-4 antibody in combination with a bispecific anti-MUC16/anti-CD3 antibody to a subject who is on a background anti-cancer therapeutic regimen. The background anti-cancer therapeutic regimen may comprise a course of administration of, e.g., a chemotherapeutic agent, or radiation. The anti-CTLA-4 antibody in combination with a bispecific anti-MUC16/anti-CD3 antibody may be added on top of the background anti-cancer therapeutic regimen. In some embodiments, the antibodies are added as part of a “background step-down” scheme, wherein the background anti-cancer therapy is gradually withdrawn from the subject over time (e.g., in a stepwise fashion) while the antibodies are administered to the subject at a constant dose, or at an increasing dose, or at a decreasing dose, over time.

In certain embodiments, the methods of the present invention comprise administering to a subject in need thereof a therapeutically effective amount of an anti-CTLA-4 antibody in combination with a therapeutically effective amount of a bispecific anti-MUC16/anti-CD3 antibody, wherein administration of the antibodies leads to increased inhibition of tumor growth. In certain embodiments, tumor growth is inhibited by at least about 10%, about 20%, about 30%, about 40%, about 50%, about 60%, about 70% or about 80% as compared to an untreated subject or a subject administered with either antibody as monotherapy. In certain embodiments, the administration of an anti-CTLA-4 antibody and a bispecific anti-MUC16/anti-CD3 antibody leads to increased tumor regression, tumor shrinkage and/or disappearance. In certain embodiments, the administration of an anti-CTLA-4 antibody and a bispecific anti-MUC16/anti-CD3 antibody leads to delay in tumor growth and development, e.g., tumor growth may be delayed by about 3 days, more than 3 days, about 7 days, more than 7 days, more than 15 days, more than 1 month, more than 3 months, more than 6 months, more than 1 year, more than 2 years, or more than 3 years as compared to an untreated subject or a subject treated with either antibody as monotherapy. In certain embodiments, administration of an anti-CTLA-4 antibody in combination with a bispecific anti-MUC16/anti-CD3 antibody prevents tumor recurrence and/or increases duration of survival of the subject, e.g., increases duration of survival by more than 15 days, more than 1 month, more than 3 months, more than 6 months, more than 12 months, more than 18 months, more than 24 months, more than 36 months, or more than 48 months than an untreated subject or a subject which is administered either antibody as monotherapy. In certain embodiments, administration of the antibodies in combination increases progression-free survival or overall survival. In certain embodiments, administration of an anti-CTLA-4 antibody in combination with a bispecific anti-MUC16/anti-CD3 antibody increases response and duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% over an untreated subject or a subject which has received either antibody as monotherapy. In certain embodiments, administration of an anti-CTLA-4 antibody and a bispecific anti-MUC16/anti-CD3 antibody to a subject with an ovarian cancer leads to complete disappearance of all evidence of tumor cells (“complete response”). In certain embodiments, administration of an anti-CTLA-4 antibody and a bispecific anti-MUC16/anti-CD3 antibody to a subject with an ovarian cancer leads to at least 30% or more decrease in tumor cells or tumor size (“partial response”). In certain embodiments, administration of an anti-CTLA-4 antibody and a bispecific anti-MUC16/anti-CD3 antibody to a subject with an ovarian cancer leads to complete or partial disappearance of tumor cells/lesions including new measurable lesions. Tumor reduction can be measured by any of the methods known in the art, e.g., X-rays, positron emission tomography (PET), computed tomography (CT), magnetic resonance imaging (MRI), cytology, histology, or molecular genetic analyses. In certain embodiments, administration of an anti-CTLA-4 antibody and a bispecific anti-MUC16/anti-CD3 antibody produces a synergistic anti-tumor effect that exceeds the combined effects of the two agents when administered alone.

In certain embodiments, the combination of administered antibodies is safe and well-tolerated by a patient wherein there is no increase in an adverse side effect (e.g., increased cytokine release (“cytokine storm”) or increased T-cell activation) as compared to a patient administered with the bispecific antibody as monotherapy.

### Anti-CTLA-4 Antibodies and Antigen-Binding Fragments Thereof

According to certain exemplary embodiments of the present invention, the methods comprise administering a therapeutically effective amount of an anti-CTLA-4 antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (vi) V_{H}-C_{H}2-C_{H}3; V_{H}-C_{L}; V_{L}-C_{H}1; (ix) V_{L}-C_{H}2; (x) V_{L}-C_{H}3; (Xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present invention using routine techniques available in the art. For example, the present invention includes methods comprising the use of bispecific antibodies wherein one arm of an immunoglobulin is specific for CTLA-4 or a fragment thereof, and the other arm of the immunoglobulin is specific for a second therapeutic target or is conjugated to a therapeutic moiety. Exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab.sup.2 bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The antibodies used in the methods of the present invention may be human antibodies. The term “human antibody,” as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present invention may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present invention specifically bind CTLA-4. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” CTLA-4, as used in the context of the present invention, includes antibodies that bind CTLA-4 or portion thereof with a K_{D} of less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human CTLA-4 may, however, have cross-reactivity to other antigens, such as CTLA-4 molecules from other (non-human) species.

According to certain exemplary embodiments of the present invention, the anti-CTLA-4 antibody, or antigen-binding fragment thereof comprises a heavy chain variable region (HCVR), light chain variable region (LCVR), and/or complementarity determining regions (CDRs) comprising any of the amino acid sequences of the anti-CTLA-4 antibodies as set forth in International Patent Publication No. WO 2019/023482. In certain exemplary embodiments, the anti-CTLA-4 antibody or antigen-binding fragment thereof that can be used in the context of the methods of the present invention comprises the heavy chain complementarity determining regions (HCDRs) of a heavy chain variable region (HCVR) comprising the amino acid sequence of SEQ ID NO: 33 and the light chain complementarity determining regions (LCDRs) of a light chain variable region (LCVR) comprising the amino acid sequence of SEQ ID NO: 34. According to certain embodiments, the anti-CTLA-4 antibody or antigen-binding fragment thereof comprises three HCDRs (HCDR1, HCDR2 and HCDR3) and three LCDRs (LCDR1, LCDR2 and LCDR3), wherein the HCDR1 comprises the amino acid sequence of SEQ ID NO: 35; the HCDR2 comprises the amino acid sequence of SEQ ID NO: 36; the HCDR3 comprises the amino acid sequence of SEQ ID NO: 37; the LCDR1 comprises the amino acid sequence of SEQ ID NO: 38; the LCDR2 comprises the amino acid sequence of SEQ ID NO: 39; and the LCDR3 comprises the amino acid sequence of SEQ ID NO: 40. In yet other embodiments, the anti-CTLA-4 antibody or antigen-binding fragment thereof comprises an HCVR comprising SEQ ID NO: 33 and an LCVR comprising SEQ ID NO: 34. In certain embodiments, the methods of the present invention comprise the use of an anti-CTLA-4 antibody, wherein the antibody comprises a heavy chain comprising the amino acid sequence of SEQ ID NO: 41. In some embodiments, the anti-CTLA-4 antibody comprises a light chain comprising the amino acid sequence of SEQ ID NO: 42. An exemplary antibody comprising an HCVR comprising SEQ ID NO: 33 and an LCVR comprising SEQ ID NO: 34 is REGN4659. According to certain exemplary embodiments, the methods of the present invention comprise the use of such an anti-CTLA-4 antibody, or a bioequivalent thereof. The term “bioequivalent”, as used herein, refers to anti-CTLA-4 antibodies or CTLA-4-binding proteins or fragments thereof that are pharmaceutical equivalents or pharmaceutical alternatives whose rate and/or extent of absorption do not show a significant difference with that of the sequence-defined antibodies when administered at the same molar dose under similar experimental conditions, either single dose or multiple dose. In the context of the invention, the term refers to antigen-binding proteins that bind to CTLA-4 which do not have clinically meaningful differences in their safety, purity and/or potency.

The anti-CTLA-4 antibodies used in the context of the methods of the present invention may have pH-dependent binding characteristics. For example, an anti-CTLA-4 antibody for use in the methods of the present invention may exhibit reduced binding to CTLA-4 at acidic pH as compared to neutral pH. Alternatively, an anti-CTLA-4 antibody of the invention may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to CTLA-4 at acidic pH as compared to neutral pH” is expressed in terms of a ratio of the K_{D} value of the antibody binding to CTLA-4 at acidic pH to the K_{D} value of the antibody binding to CTLA-4 at neutral pH (or vice versa). For example, an antibody or antigen-binding fragment thereof may be regarded as exhibiting “reduced binding to CTLA-4 at acidic pH as compared to neutral pH” for purposes of the present invention if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. As used herein, the expression “acidic pH” means a pH of 6.0 or less.

### Bispecific Anti-MUC16/Anti-CD3 Antibodies

According to certain exemplary embodiments of the present invention, the methods comprise administering a therapeutically effective amount of a bispecific antibody that specifically binds CD3 and MUC16. Such antibodies may be referred to herein as, e.g., “anti-MUC16/anti-CD3,” or “anti-MUC16×CD3” or “MUC16×CD3” bispecific antibodies, or other similar terminology.

As used herein, the expression “bispecific antibody” refers to an immunoglobulin protein comprising at least a first antigen-binding domain and a second antigen-binding domain. In the context of the present invention, the first antigen-binding domain specifically binds a first antigen (e.g., MUC16), and the second antigen-binding domain specifically binds a second, distinct antigen (e.g., CD3). Each antigen-binding domain of a bispecific antibody comprises a heavy chain variable domain (HCVR) and a light chain variable domain (LCVR), each comprising three CDRs. In the context of a bispecific antibody, the CDRs of the first antigen-binding domain may be designated with the prefix “A” and the CDRs of the second antigen-binding domain may be designated with the prefix “B”. Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain are each connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present invention, the multimerizing component is an Fc portion of an immunoglobulin (comprising a C_{H2}-C_{H3} domain), e.g., an Fc domain of an IgG selected from the isotypes IgG1, IgG2, IgG3, and IgG4, as well as any allotype within each isotype group.

Bispecific antibodies of the present invention typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab₂ bispecific formats (see, e.g., Klein et al. 2012, mAbs 4:6, 1-11, and references cited therein, for a review of the foregoing formats).

In the context of bispecific antibodies of the present invention, Fc domains may comprise one or more amino acid changes (e.g., insertions, deletions or substitutions) as compared to the wild-type, naturally occurring version of the Fc domain. For example, the invention includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H2} or a C_{H3} region, wherein the modification increases the affinity of the Fc domain to FcRn in an acidic environment (e.g., in an endosome where pH ranges from about 5.5 to about 6.0). Non-limiting examples of such Fc modifications are disclosed in US Patent Publication No. 20150266966, incorporated herein in its entirety.

The present invention also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 CH2]-[IgG4 CH3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 CH2]-[IgG1 CH3]. These and other examples of chimeric Fc domains that can be included in any of the antigen-binding molecules of the present invention are described in US Patent Publication No. 20140243504, which is herein incorporated in its entirety. Chimeric Fc domains having these general structural arrangements, and variants thereof, can have altered Fc receptor binding, which in turn affects Fc effector function.

According to certain exemplary embodiments of the present invention, the bispecific anti-MUC16/anti-CD3 antibody, or antigen-binding fragment thereof comprises heavy chain variable regions (A-HCVR and B-HCVR), light chain variable regions (A-LCVR and B-LCVR), and/or complementarity determining regions (CDRs) comprising any of the amino acid sequences of the bispecific anti-MUC16/anti-CD3 antibodies as set forth in US Patent Publication No. 20180112001. In certain exemplary embodiments, the bispecific anti-MUC16/anti-CD3 antibody or antigen-binding fragment thereof that can be used in the context of the methods of the present invention comprises: (a) a first antigen-binding arm comprising the heavy chain complementarity determining regions (A-HCDR1, A-HCDR2 and A-HCDR3) of a heavy chain variable region (A-HCVR) comprising the amino acid sequence of SEQ ID NO: 1 and the light chain complementarity determining regions (A-LCDR1, A-LCDR2 and A-LCDR3) of a light chain variable region (A-LCVR) comprising the amino acid sequence of SEQ ID NO: 2; and (b) a second antigen-binding arm comprising the heavy chain CDRs (B-HCDR1, B-HCDR2 and B-HCDR3) of a HCVR (B-HCVR) comprising an amino acid sequence of SEQ ID NO: 3, SEQ ID NO: 4, SEQ ID NO: 5, SEQ ID NO: 6 or SEQ ID NO: 7, and the light chain CDRs (B-LCDR1, B-LCDR2 and B-LCDR3) of a LCVR (B-LCVR) comprising the amino acid sequence of SEQ ID NO: 2. According to certain embodiments, the A-HCDR1 comprises the amino acid sequence of SEQ ID NO: 8; the A-HCDR2 comprises the amino acid sequence of SEQ ID NO: 9; the A-HCDR3 comprises the amino acid sequence of SEQ ID NO: 10; the A-LCDR1 comprises the amino acid sequence of SEQ ID NO: 11; the A-LCDR2 comprises the amino acid sequence of SEQ ID NO: 12; the A-LCDR3 comprises the amino acid sequence of SEQ ID NO: 13; the B-HCDR1 comprises the amino acid sequence of SEQ ID NO: 14, SEQ ID NO: 17, SEQ ID NO: 20, SEQ ID NO: 23, or SEQ ID NO: 26; the B-HCDR2 comprises the amino acid sequence of SEQ ID NO: 15, SEQ ID NO: 18, SEQ ID NO: 21, SEQ ID NO: 24, or SEQ ID NO: 27; and the B-HCDR3 comprises the amino acid sequence of SEQ ID NO: 16, SEQ ID NO: 19, SEQ ID NO: 22, SEQ ID NO: 25, or SEQ ID NO: 28; and the B-LCDR1 comprises the amino acid sequence of SEQ ID NO: 11; the B-LCDR2 comprises the amino acid sequence of SEQ ID NO: 12; the B-LCDR3 comprises the amino acid sequence of SEQ ID NO: 13. In yet other embodiments, the bispecific anti-MUC16/anti-CD3 antibody or antigen-binding fragment thereof comprises: (a) a first antigen-binding arm comprising a HCVR (A-HCVR) comprising SEQ ID NO: 1 and a LCVR (A-LCVR) comprising SEQ ID NO: 2; and (b) a second antigen-binding arm comprising a HCVR (B-HCVR) comprising SEQ ID NO: 3, SEQ ID NO: 4, SEQ ID NO: 5, SEQ ID NO: 6, or SEQ ID NO: 7, and a LCVR (B-LCVR) comprising SEQ ID NO: 2. In certain exemplary embodiments, the bispecific anti-CD3×MUC16 antibody comprises a MUC16-binding arm comprising a heavy chain comprising the amino acid sequence of SEQ ID NO: 29 and a light chain comprising the amino acid sequence of SEQ ID NO: 30, and a CD3-binding arm comprising a heavy chain comprising the amino acid sequence of SEQ ID NO: 31 and a light chain comprising the amino acid sequence of SEQ ID NO: 30. In certain exemplary embodiments, the bispecific anti-CD3×MUC16 antibody comprises a MUC16-binding arm comprising a heavy chain comprising the amino acid sequence of SEQ ID NO: 29 and a light chain comprising the amino acid sequence of SEQ ID NO: 30, and a CD3-binding arm comprising a heavy chain comprising the amino acid sequence of SEQ ID NO: 32 and a light chain comprising the amino acid sequence of SEQ ID NO: 30.

Other bispecific anti-MUC16/anti-CD3 antibodies that can be used in the context of the methods of the present invention include, e.g., any of the antibodies as set forth in International Patent Publication No. WO 2018/067331.

### Combination Therapies

The methods of the present invention, according to certain embodiments, comprise administering to the subject an anti-MUC16/anti-CD3 bispecific antibody in combination with an anti-CTLA-4 antibody. In certain embodiments, the methods of the present invention comprise administering the antibodies for additive or synergistic activity to treat cancer, preferably an ovarian cancer. As used herein, the expression “in combination with” means that the anti-MUC16/anti-CD3 bispecific antibody is administered before, after, or concurrent with the anti-CTLA-4 antibody. The term “in combination with” also includes sequential or concomitant administration of anti-CTLA-4 antibody and a bispecific anti-MUC16/anti-CD3 antibody. For example, when administered “before” the bispecific anti-MUC16/anti-CD3 antibody, the anti-CTLA-4 antibody may be administered more than 150 hours, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the bispecific anti-MUC16/anti-CD3 antibody. When administered “after” the bispecific anti-MUC16/anti-CD3 antibody, the anti-CTLA-4 antibody may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, or more than 72 hours after the administration of the bispecific anti-MUC16/anti-CD3 antibody. Administration “concurrent” with the bispecific anti-MUC16/anti-CD3 antibody means that the anti-CTLA-4 antibody is administered to the subject in a separate dosage form within less than 5 minutes (before, after, or at the same time) of administration of the bispecific anti-MUC16/anti-CD3 antibody, or administered to the subject as a single combined dosage formulation comprising both the anti-CTLA-4 antibody and the bispecific anti-MUC16/anti-CD3 antibody.

In certain embodiments, the methods of the present invention comprise administration of a third therapeutic agent wherein the third therapeutic agent is an anti-cancer drug. As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

In certain embodiments, the methods of the present invention comprise administration of a third therapeutic agent selected from the group consisting of radiation, surgery, a cancer vaccine, a PD-1 inhibitor (e.g., an anti-PD-1 antibody), a PD-L1 inhibitor (e.g., an anti-PD-L1 antibody), a LAG-3 inhibitor, a TIM3 inhibitor, a BTLA inhibitor, a TIGIT inhibitor, a CD47 inhibitor, an antagonist of another T-cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGF.beta.) inhibitor, an epidermal growth factor receptor (EGFR) inhibitor (e.g., erlotinib, cetuximab), an agonist to a co-stimulatory receptor (e.g., an agonist to glucocorticoid-induced TNFR-related protein), an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin, a cancer vaccine), an adjuvant to increase antigen presentation (e.g., granulocyte-macrophage colony-stimulating factor), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, an antibody-drug conjugate (ADC) (e.g., anti-CD19-DM4 ADC, and anti-DS6-DM4 ADC), chimeric antigen receptor T cells (e.g., CD19-targeted T cells), an anti-inflammatory drug (e.g., corticosteroids, and non-steroidal anti-inflammatory drugs), and a dietary supplement such as anti-oxidants.

In certain embodiments, the methods of the invention comprise administering an anti-CTLA-4 antibody and an anti-MUC16/anti-CD3 bispecific antibody in combination with radiation therapy to generate long-term durable anti-tumor responses and/or enhance survival of patients with cancer.

In some embodiments, the methods of the invention comprise administering radiation therapy prior to, concomitantly or after administering an anti-CTLA-4 antibody and a bispecific anti-MUC16/anti-CD3 antibody to a cancer patient. For example, radiation therapy may be administered in one or more doses to tumor lesions after administration of one or more doses of the antibodies. In some embodiments, radiation therapy may be administered locally to a tumor lesion to enhance the local immunogenicity of a patient's tumor (adjuvinating radiation) and/or to kill tumor cells (ablative radiation) after systemic administration of an anti-CTLA-4 antibody and/or a bispecific anti-MUC16/anti-CD3 antibody. In certain embodiments, the antibodies may be administered in combination with radiation therapy and a chemotherapeutic agent (e.g., carboplatin and/or paclitaxel) or a VEGF antagonist (e.g., aflibercept).

### Pharmaceutical Compositions and Administration

The present invention includes methods which comprise administering an anti-CTLA-4 antibody in combination with a bispecific anti-MUC16/anti-CD3 antibody to a subject wherein the antibodies are contained within separate or combined (single) pharmaceutical composition. The pharmaceutical compositions of the invention may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Administration Regimens

The present invention includes methods comprising administering to a subject an anti-CTLA-4 antibody at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments, the present invention includes methods comprising administering to a subject a bispecific anti-MUC16/anti-CD3 antibody at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments, the methods involve the administration of an anti-CTLA-4 antibody in combination with a bispecific anti-MUC16/anti-CD3 antibody at a dosing frequency of about four times a week, twice a week, once a week, once every two weeks, once every three weeks, once every four weeks, once every five weeks, once every six weeks, once every eight weeks, once every nine weeks, once every twelve weeks, or less frequently so long as a therapeutic response is achieved.

According to certain embodiments of the present invention, multiple doses of an anti-CTLA-4 antibody in combination with a bispecific anti-MUC16/anti-CD3 antibody may be administered to a subject over a defined time course. The methods according to this aspect of the invention comprise sequentially administering to a subject one or more doses of an anti-CTLA-4 antibody in combination with one or more doses of a bispecific anti-MUC16/anti-CD3 antibody. As used herein, “sequentially administering” means that each dose of the antibody is administered to the subject at a different point in time, e.g., on different days separated by a predetermined interval (e.g., hours, days, weeks or months). The present invention includes methods which comprise sequentially administering to the patient a single initial dose of an anti-CTLA-4 antibody, followed by one or more secondary doses of the anti-CTLA-4 antibody, and optionally followed by one or more tertiary doses of the anti-CTLA-4 antibody. In certain embodiments, the methods further comprise sequentially administering to the patient a single initial dose of a bispecific anti-MUC16/anti-CD3 antibody, followed by one or more secondary doses of the bispecific antibody, and optionally followed by one or more tertiary doses of the bispecific antibody.

According to certain embodiments of the present invention, multiple doses of an anti-CTLA-4 antibody and a bispecific anti-MUC16/anti-CD3 antibody may be administered to a subject over a defined time course. The methods according to this aspect of the invention comprise sequentially administering to a subject multiple doses of an anti-CTLA-4 antibody and a bispecific anti-MUC16/anti-CD3 antibody. As used herein, “sequentially administering” means that each dose of the anti-CTLA-4 antibody in combination with the bispecific antibody is administered to the subject at a different point in time, e.g., on different days separated by a predetermined interval (e.g., hours, days, weeks or months).

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antibody (anti-CTLA-4 antibody or bispecific antibody). In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, an anti-CTLA-4 antibody may be administered to a patient with an ovarian cancer at a loading dose of about 1-3 mg/kg followed by one or more maintenance doses of about 0.1 to about 20 mg/kg of the patient's body weight.

In one exemplary embodiment of the present invention, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of anti-CTLA-4 antibody (and/or bispecific anti-MUC16/anti-CD3 antibody) which is administered to a patient prior to the administration of the very next dose in the sequence with no intervening doses.

The methods according to this aspect of the invention may comprise administering to a patient any number of secondary and/or tertiary doses of an anti-CTLA-4 antibody (and/or bispecific anti-MUC16/anti-CD3 antibody). For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In certain embodiments, one or more doses of an anti-CTLA-4 antibody and/or a bispecific anti-MUC16/anti-CD3 antibody are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

The present invention includes methods comprising sequential administration of an anti-CTLA-4 antibody in combination with a bispecific anti-MUC16/anti-CD3 antibody, to a patient to treat an ovarian cancer (e.g., serous cancer). In some embodiments, the present methods comprise administering one or more doses of an anti-CTLA-4 antibody followed by one or more doses of a bispecific anti-MUC16/anti-CD3 antibody. In certain embodiments, the present methods comprise administering a single dose of an anti-CTLA-4 antibody followed by one or more doses of a bispecific anti-MUC16/anti-CD3 antibody. In some embodiments, one or more doses of about 0.1 mg/kg to about 20 mg/kg of an anti-CTLA-4 antibody may be administered followed by one or more doses of about 0.1 mg/kg to about 20 mg/kg of the bispecific antibody to inhibit tumor growth and/or to prevent tumor recurrence in a subject with an ovarian cancer. In some embodiments, the anti-CTLA-4 antibody is administered at one or more doses followed by one or more doses of the bispecific antibody resulting in increased anti-tumor efficacy (e.g., greater inhibition of tumor growth, increased prevention of tumor recurrence as compared to an untreated subject or a subject administered with either antibody as monotherapy). Alternative embodiments of the invention pertain to concomitant administration of anti-CTLA-4 antibody and the bispecific antibody which is administered at a separate dosage at a similar or different frequency relative to the anti-CTLA-4 antibody. In some embodiments, the bispecific antibody is administered before, after or concurrently with the anti-CTLA-4 antibody. In certain embodiments, the bispecific antibody is administered as a single dosage formulation with the anti-CTLA-4 antibody.

### Dosage

The amount of anti-CTLA-4 antibody and/or bispecific anti-MUC16/anti-CD3 antibody administered to a subject according to the methods of the present invention is, generally, a therapeutically effective amount. As used herein, the phrase “therapeutically effective amount” means an amount of antibody (anti-CTLA-4 antibody or bispecific anti-MUC16/anti-CD3 antibody) that results in one or more of: (a) a reduction in the severity or duration of a symptom of a cancer (e.g., ovarian cancer); (b) inhibition of tumor growth, or an increase in tumor necrosis, tumor shrinkage and/or tumor disappearance; (c) delay in tumor growth and development; (d) inhibit or retard or stop tumor metastasis; (e) prevention of recurrence of tumor growth; (f) increase in survival of a subject with cancer (e.g., ovarian cancer); and/or (g) a reduction in the use or need for conventional anti-cancer therapy (e.g., reduced or eliminated use of chemotherapeutic or cytotoxic agents) as compared to an untreated subject or a subject administered with either antibody as monotherapy.

In the case of an anti-CTLA-4 antibody (or antigen-binding fragment), a therapeutically effective amount can be from about 0.05 mg to about 1500 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, or about 1500 mg, of the anti-CTLA-4 antibody. In some cases, the therapeutically effective amount of the anti-CTLA4 antibody (or antigen-binding fragment) may be from about 0.5 mg/kg to about 2.5 mg/kg, or from about 20 mg to about 250 mg.

In the case of a bispecific anti-MUC16/anti-CD3 antibody, a therapeutically effective amount can be from about 10 mg to 1500 mg, e.g., about 10 mg, about 20 mg, about 50 mg, about 70 mg, about 100 mg, about 120 mg, about 150 mg, about 200 mg, about 250 mg, about 300 mg, about 350 mg, about 400 mg, about 450 mg, about 500 mg, about 550 mg, about 600 mg, about 700 mg, about 800 mg, about 900 mg, about 1000 mg, about 1050 mg, about 1100 mg, about 1200 mg, about 1300 mg, about 1400 mg, or about 1500 mg of the bispecific anti-MUC16/anti-CD3 antibody. In some cases, the therapeutically effective amount of the anti-MUC16×CD3 bispecific antibody may be from about 30 mg to about 900 mg.

The amount of either anti-CTLA-4 antibody or bispecific anti-MUC16/anti-CD3 antibody contained within the individual doses may be expressed in terms of milligrams of antibody per kilogram of subject body weight (i.e., mg/kg). In certain embodiments, either anti-CTLA-4 antibody or bispecific anti-MUC16/anti-CD3 antibody used in the methods of the present invention may be administered to a subject at a dose of about 0.0001 to about 100 mg/kg of subject body weight. For example, anti-CTLA-4 antibody may be administered at dose of about 0.1 mg/kg to about 20 mg/kg of a patient's body weight. The bispecific anti-MUC16/anti-CD3 antibody may be administered at a dose of about 0.1 mg/kg to about 20 mg/kg of a patient's body weight.

A summary of the sequences and the corresponding SEQ ID NOs referenced herein is shown in Table 1, below.

TABLE 1
Summary of Sequences

| SEQ ID NO: | Description |
|---|---|
| 1 | Anti-MUC16 Heavy Chain Variable Region |
| 2 | Anti-MUC16 and Anti-CD3 Light Chain Variable Region |
| 3 | Anti-CD3-G Heavy Chain Variable Region |
| 4 | Anti-CD3-G5 Heavy Chain Variable Region |
| 5 | Anti-CD3-G9 Heavy Chain Variable Region |
| 6 | Anti-CD3-G10 Heavy Chain Variable Region |
| 7 | Anti-CD3-G20 Heavy Chain Variable Region |
| 8 | Anti-MUC16 HCDR1 |
| 9 | Anti-MUC16 HCDR2 |
| 10 | Anti-MUC16 HCDR3 |
| 11 | Anti-MUC16 and Anti-CD3 LCDR1 |
| 12 | Anti-MUC16 and Anti-CD3 LCDR2 |
| 13 | Anti-MUC16 and Anti-CD3 LCDR3 |
| 14 | Anti-CD3-G HCDR1 |
| 15 | Anti-CD3-G HCDR2 |
| 16 | Anti-CD3-G HCDR3 |
| 17 | Anti-CD3-G5 HCDR1 |
| 18 | Anti-CD3-G5 HCDR2 |
| 19 | Anti-CD3-G5 HCDR3 |
| 20 | Anti-CD3-G9 HCDR1 |
| 21 | Anti-CD3-G9 HCDR2 |
| 22 | Anti-CD3-G9 HCDR3 |
| 23 | Anti-CD3-G10 HCDR1 |
| 24 | Anti-CD3-G10 HCDR2 |
| 25 | Anti-CD3-G10 HCDR3 |
| 26 | Anti-CD3-G20 HCDR1 |
| 27 | Anti-CD3-G20 HCDR2 |
| 28 | Anti-CD3-G20 HCDR3 |
| 29 | Anti-MUC16 Heavy Chain |
| 30 | Anti-MUC16 and Anti-CD3 Light Chain |
| 31 | Anti-CD3-G Heavy Chain |
| 32 | Anti-CD3-G20 Heavy Chain |
| 33 | Anti-CTLA-4 Heavy Chain Variable Region |
| 34 | Anti-CTLA-4 Light Chain Variable Region |
| 35 | Anti-CTLA-4 HCDR1 |
| 36 | Anti-CTLA-4 HCDR2 |
| 37 | Anti-CTLA-4 HCDR3 |
| 38 | Anti-CTLA-4 LCDR1 |
| 39 | Anti-CTLA-4 LCDR2 |
| 40 | Anti-CTLA-4 LCDR3 |
| 41 | Anti-CTLA-4 Heavy Chain |
| 42 | Anti-CTLA-4 Light Chain |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Bispecific Antibodies that Bind MUC16 and CD3

The present invention provides bispecific antigen-binding molecules that bind CD3 and MUC16; such bispecific antigen-binding molecules are also referred to herein as “anti-MUC16/anti-CD3 or anti-MUC16×CD3 bispecific molecules.” The anti-MUC16 portion of the anti-MUC16/anti-CD3 bispecific molecule is designed for targeting tumor cells that express MUC16 (also known as CA-125), and the anti-CD3 portion of the bispecific molecule is designed to activate T-cells. The simultaneous binding of MUC16 on a tumor cell and CD3 on a T-cell facilitates directed killing (cell lysis) of the targeted tumor cell by the activated T-cell.

Bispecific antibodies comprising an anti-MUC16-specific binding domain and an anti-CD3-specific binding domain were constructed using standard methodologies, wherein the anti-MUC16 antigen binding domain and the anti-CD3 antigen binding domain each comprise different, distinct HCVRs paired with a common LCVR. In exemplified bispecific antibodies, the molecules were constructed utilizing a heavy chain from an anti-CD3 antibody, a heavy chain from an anti-MUC16 antibody and a common light chain from the anti-MUC16 antibody. In other instances, the bispecific antibodies may be constructed utilizing a heavy chain from an anti-CD3 antibody, a heavy chain from an anti-MUC16 antibody and a light chain from an anti-CD3 antibody or an antibody light chain known to be promiscuous or pair effectively with a variety of heavy chain arms.

Exemplified bispecific antibodies were manufactured having an IgG1 Fc domain (BSMUC16/CD3-001, -002, -003, and -004) or a modified (chimeric) IgG4 Fc domain (BSMUC16/CD3-005) as set forth in US Patent Application Publication No. US20140243504A1, published on Aug. 28, 2014.

A summary of the component parts of the antigen-binding domains of the various anti-MUC16×CD3 bispecific antibodies constructed is set forth in Table 2.

TABLE 2
Summary of Component Parts of Anti-
MUC16 × CD3 Bispecific Antibodies

| | Anti-MUC16 | Anti-CD3 | |
|---|---|---|---|
| | Antigen-Binding | Antigen-Binding | |
| Bispecific | Domain | Domain | Common |
| Antibody | Heavy Chain | Heavy Chain | Light Chain |
| Identifier | Variable Region | Variable Region | Variable Region |
| BSMUC16/ | (SEQ ID NO: 1) | CD3-VH-G | (SEQ ID NO: 2) |
| CD3-001 | | (SEQ ID NO: 3) | |
| BSMUC16/ | (SEQ ID NO: 1) | CD3-VH-G5 | (SEQ ID NO: 2) |
| CD3-002 | | (SEQ ID NO: 4) | |
| BSMUC16/ | (SEQ ID NO: 1) | CD3-VH-G9 | (SEQ ID NO: 2) |
| CD3-003 | | (SEQ ID NO: 5) | |
| BSMUC16/ | (SEQ ID NO: 1) | CD3-VH-G10 | (SEQ ID NO: 2) |
| CD3-004 | | (SEQ ID NO: 6) | |
| BSMUC16/ | (SEQ ID NO: 1) | CD3-VH-G20 | (SEQ ID NO: 2) |
| CD3-005 | | (SEQ ID NO: 7) | |

### Example 2: CTLA-4 Blockade Enhances Anti-Tumor Activity of Anti-MUC16×CD3 Bispecific Antibodies in Syngeneic Tumor Models

The in vivo efficacy of an anti-MUC16/anti-CD3 (BSMUC16/CD3-001, also referred to herein as REGN4018) bispecific antibody in combination with CTLA-4 blockade was evaluated in a syngeneic tumor model in three experiments.

For these experiments, an immune-competent mouse was engineered so that the murine CD3 gene was replaced with human CD3 and a portion of the mouse MUC16 gene was replaced with the human sequence. The replacements resulted in a mouse whose T cells express human CD3 and that expresses a chimeric MUC16 molecule containing a portion of human MUC16 where the REGN4018 bispecific antibody binds.

In all three experiments, the ID8-VEGF cell line (mouse ovarian cancer) engineered to express the portion of human MUC16 was used. The anti-CTLA-4 antibody used in these experiments was derived from a commercially available murine antibody (clone 9D9, Bio X Cell) in which mIgG2b was replaced with mIgG2a isotype. The anti-CTLA-4 antibody with mIgG2a isotype and mIgG2a isotype control were generated at Regeneron (Tarrytown, N.Y.).

### A. Experiment 1

In the first experiment, mice were implanted with ID8-VEGF/huMUC16 cells SC at 10⁷ cells per mouse. Mice were randomized on Day 4, with the 60 mm³ average tumor volume in each group. Mice were treated with REGN4018 (5 mg/kg, IP) or treated with CD3-binding control (5 mg/kg, IP) in combination with anti-CTLA-4 antibody (5 mg/kg, IP) or mIgG2a isotype control (5 mg/kg, IP) starting on Day 4 post tumor implantation and then on Days 6, 10, 14, 17.

Data shown in Table 3 represent a mean tumor volume assessed on Day 39 post tumor implantation, the last day when all mice are alive. At later time points, mice with tumor burden exceeding the maximum allowed by IACUC were sacrificed. Statistical significance was determined by one-way ANOVA (non-parametric) with Kruskal-Wallis comparison test. Treatment with a combination of REGN4018 and anti-CTLA-4 antibody resulted in significant inhibition of tumor growth compared to the combination of CD3-binding control and isotype control (p<0.01), while treatment with REGN4018 monotherapy or anti-CTLA-4 antibody monotherapy did not result in tumor growth inhibition. By Day 61, the last time point of the experiment, there were 3 tumor free mice in the combination of REGN4018 and anti-CTLA-4 antibody group.

TABLE 3
Tumor Volumes on Day 39 post tumor implantation

| | | Average tumor | |
|---|---|---|---|
| | | volume (mm³) | Tumor |
| | Number | on Day 39 | free on |
| Antibody (mg/kg) | of mice | (Mean +/− SEM) | Day 61 |
| CD3-binding control (5) + | 9 | 275.0 +/− 47.6 | 0 |
| isotype control (5) | | | |
| CD3-binding control (5) + | 10 | 378.6 +/− 65.7 | 1 |
| anti-CTLA-4 (5) | | | |
| REGN4018 (5) + isotype | 10 | 257.3 +/− 61.6 | 0 |
| control (5) | | | |
| REGN4018 (5) + anti-CTLA-4 | 9 | 66.7 +/− 26.2 | 3 |
| (5) | | | |

### B. Experiment 2

In the second experiment, the effect of REGN4018 antibody in combination with anti-CTLA-4 antibody of lower doses (2.5 mg/kg or 0.5 mg/kg) on tumor growth was tested in the same syngeneic tumor model. Mice were implanted with ID8-VEGF/huMUC16 cells SC at 10⁷ cells per mouse and randomized on Day 5, with the 80 mm³ average tumor volume in each group (N=8 mice/group). Mice were administered REGN4018 (5 mg/kg, IP) or administered CD3-binding control (5 mg/kg, IP) in combination with anti-CTLA-4 antibody (2.5 mg/kg or 0.5 mg/kg IP) or isotype control (2.5 mg/kg, IP) starting on Day 5 post tumor implantation and then on Days 7, 11, 15, 18.

Mean tumor volumes of each group at Day 34, the last time point of tumor measurements, are presented in Table 4. Statistical significance was determined by one-way ANOVA (non-parametric) with Kruskal-Wallis comparison test. Treatment with a combination of REGN4018 at 5 mg/kg and anti-CTLA-4 antibody at either 2.5 mg/kg or 0.5 mg/kg resulted in significant inhibition of tumor growth compared to the combination of CD3-binding control and isotype control (p<0.001), while treatment with REGN4018 monotherapy or anti-CTLA-4 antibody monotherapy did not result in tumor growth inhibition.

TABLE 4
Tumor Volumes on Day 34 post tumor implantation

| | Average tumor volume |
|---|---|
| | (mm³) on Day 34 |
| Antibody (mg/kg) | (Mean +/− SEM) |
| CD3-binding control (5) + isotype control (2.5) | 330.5 +/− 36.4 |
| CD3-binding control (5) + anti-CTLA-4 (2.5) | 564.7 +/− 62.9 |
| REGN4018 (5) + isotype control (2.5) | 250.6 +/− 52.0 |
| REGN4018 (5) + anti-CTLA-4 (0.5) | 37.5 +/− 9.6 |
| REGN4018 (5) + anti-CTLA-4 (2.5) | 55.4 +/− 23.0 |

### C. Experiment 3

In the third experiment in the ascites tumor model, mice were injected intraperitoneally (IP) with ID8-VEGF/huMUC16 cells at 5×10⁵ on Day 0 and mice were randomized by weight (n=8 per group). Mice were treated with REGN4018 (5 mg/kg, IP) or treated with CD3-binding control (5 mg/kg, IP) in combination with anti-CTLA-4 antibody (5 mg/kg, IP) or isotype control (5 mg/kg, IP) on Days 3, 6, 9, 12, 15 post tumor implantation. Survival in this experiment was defined by the maximum allowed tumor weight gain due to tumor growth. Accordingly, the slower weight gain in this experiment reflects better survival. Mice were sacrificed when they had a weight-gain of more than 20% due to ascites-induced abdominal distension. As shown in Table 5, treatment with a combination of REGN4018 and anti-CTLA-4 antibody extended the median survival to 50 days compared to the median survival of 34 days seen in the group that received the combination of CD3-binding control and isotype control (p<0.001, Mantel-Cox method), indicating significant anti-tumor efficacy of a combination treatment with REGN4018 and anti-CTLA4 antibody.

In order to ensure that the increase in median survival of mice in the group receiving the combination of REGN4018 plus anti-CTLA4 was due to increased efficacy of the combination therapy itself and not due to toxicity of the combination, a control experiment was done. In this control experiment, naïve non-tumor bearing mice were treated with REGN4018 (5 mg/kg, IP) and anti-CTLA-4 antibody (5 mg/kg, IP) using the same treatment schedule noted above. The results showed that the combination treatment did not induce weight loss in naïve non-tumor-bearing mice (data not shown). This confirms that the weight loss in tumor bearing mice was a result of increased efficacy of the combination therapy, which resulted in a reduction of tumor weight and not due to toxicity of the combination therapy.

TABLE 5
Median Survival in the ID8-VEGF/huMUC16
Ascites Tumor Model

| Antibody (mg/kg) | Median survival (Days) |
|---|---|
| CD3-binding control (5) + isotype control (5) | 34 |
| CD3-binding control (5) + anti-CTLA-4 (5) | 34 |
| REGN4018 (5) + isotype control (5) | 39 |
| REGN4018 (5) + anti-CTLA-4 (5) | 50 |

### Example 3: Assessment of Serum Cytokine Induction in Tumor-Bearing Mice

Because cytokine release syndrome is a frequent serious side effect of CD3 bispecific or anti-CTLA-4 antibody treatment in humans, we assessed T cell activation by measuring serum cytokines following treatment with REGN4018 in combination with anti-CTLA-4 antibody in genetically humanized MUC16/CD3 naïve non-tumor-bearing mice. Serum samples were collected 4 hours after treatment with a single dose of REGN4018 (5 mg/kg, IP) or CD3-binding control (5 mg/kg, IP) in combination with anti-CTLA-4 antibody (5 mg/kg, IP) or isotype control (5 mg/kg, IP). Serum cytokine levels of interleukin-2 (IL-2), IL-6, interferon γ (IFNγ), IL-10, IL-12p70, C-X-C Motif Chemokine Ligand 1 (CXCL1), tumor necrosis factor α (TNFα), IL-5, and IL-1β were analyzed using V-PLEX ProInflammatory Panel I Human Kit following the manufacturer's instructions (Meso Scale Discovery, Rockville, Mass.). Cytokines were measured in two separate studies with 5 mice per group.

Single-dose combination of REGN4018 (5 mg/kg) and anti-CTLA-4 antibody (5 mg/kg), as well REGN4018 (5 mg/kg), or anti-CTLA-4 antibody (5 mg/kg) monotherapy did not cause significant serum cytokine increase in humanized MUC16/CD3 naïve mice at 4 hours post administration compared to controls (data not shown).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

TABLE 6
Sequences Excluded from ST. 26-Formatted Sequence Listing

| SEQ ID NO: | Sequence |
|---|---|
| 12 | TAS |
| 39 | AAS |

1. A method of treating or inhibiting the growth of a MUC16-expressing tumor comprising administering to a subject in need thereof (a) a therapeutically effective amount of a bispecific antibody comprising a first antigen-binding arm that specifically binds MUC16 and a second antigen-binding arm that specifically binds CD3; and (b) a therapeutically effective amount of an antibody or antigen-binding fragment thereof that specifically binds cytotoxic T-lymphocyte-associated protein 4 (CTLA-4).

2. The method of claim 1, wherein the MUC16-expressing tumor is selected from an ovarian cancer, ovarian serous carcinoma, a breast cancer, invasive lobular breast carcinoma, a pancreatic cancer, pancreatic ductal adenocarcinoma, a bladder cancer, or non-small cell lung cancer.

3. The method of claim 1, wherein MUC16-expressing tumor is an ovarian cancer or ovarian serous carcinoma.

4. The method of claim 1, wherein the subject is resistant to, or inadequately responsive to, or relapsed after, prior therapy.

5. The method of claim 1, wherein the bispecific antibody is administered at a dose of between 0.1 mg/kg and 20 mg/kg of the subject's body weight.

6. The method of claim 1, wherein the anti-CTLA4 antibody or antigen-binding fragment thereof is administered at a dose of between 0.1 mg/kg and 20 mg/kg of the subject's body weight.

7. The method of claim 1, wherein the anti-CTLA4 antibody or antigen-binding fragment thereof and/or the bispecific antibody is administered at a dose of between 10 mg and 1500 mg.

8. The method of claim 1, wherein one or more doses of the bispecific antibody are administered with one or more doses of the anti-CTLA4 antibody or antigen-binding fragment thereof.

9. The method of claim 8, wherein a subsequent dose of the anti-CTLA4 antibody or antigen-binding fragment thereof and/or the bispecific antibody is administered 0.5-12 weeks after an immediately preceding dose.

10. The method of claim 1, wherein the anti-CTLA-4 antibody or antigen-binding fragment thereof is administered prior to, or after the bispecific antibody.

11. The method of claim 1, wherein the anti-CTLA4 antibody or antigen-binding fragment thereof and/or the bispecific antibody is administered intravenously, subcutaneously, or intraperitoneally.

12. The method of claim 1, further comprising administering to the subject a third therapeutic agent or therapy.

13. The method of claim 12, wherein the third therapeutic agent or therapy is selected from the group consisting of radiation, surgery, a chemotherapeutic agent, a cancer vaccine, a PD1 inhibitor, a PD-L1 inhibitor, a LAG-3 inhibitor, a TIM3 inhibitor, a BTLA inhibitor, a TIGIT inhibitor, a CD47 inhibitor, an indoleamine-2,3-dioxygenase (IDO) inhibitor, a vascular endothelial growth factor (VEGF) antagonist, an angiopoietin-2 (Ang2) inhibitor, a transforming growth factor beta (TGF.beta.) inhibitor, an epidermal growth factor receptor (EGFR) inhibitor, an antibody to a tumor-specific antigen, Bacillus Calmette-Guerin vaccine, granulocyte-macrophage colony-stimulating factor, a cytotoxin, an interleukin 6 receptor (IL-6R) inhibitor, an interleukin 4 receptor (IL-4R) inhibitor, an IL-10 inhibitor, IL-2, IL-7, IL-21, IL-15, an antibody-drug conjugate, an anti-inflammatory drug, and a dietary supplement.

14. The method of claim 1, wherein the anti-CTLA4 antibody or antigen-binding fragment thereof is selected from ipilimumab and REGN4659.

15. The method of claim 1, wherein the anti-CTLA-4 antibody or antigen-binding fragment thereof comprises three heavy chain complementarity determining regions (HCDR1, HCDR2 and HCDR3) of a heavy chain variable region (HCVR) comprising the amino acid sequence of SEQ ID NO: 33, and three light chain complementarity determining regions (LCDR1, LCDR2 and LCDR3) of a light chain variable region (LCVR) comprising the amino acid sequence of SEQ ID NO: 34.

16. The method of claim 15, wherein HCDR1 comprises the amino acid sequence of SEQ ID NO: 35; HCDR2 comprises the amino acid sequence of SEQ ID NO: 36; HCDR3 comprises the amino acid sequence of SEQ ID NO: 37; LCDR1 comprises the amino acid sequence of SEQ ID NO: 38; LCDR2 comprises the amino acid sequence of SEQ ID NO: 39; and LCDR3 comprises the amino acid sequence of SEQ ID NO: 40.

17. The method of claim 16, wherein the anti-CTLA4 antibody or antigen-binding fragment thereof comprises a HCVR comprising the amino acid sequence of SEQ ID NO: 33, and a LCVR comprising the amino acid sequence of SEQ ID NO: 34.

18. The method of claim 15, wherein the anti-CTLA-4 antibody comprises a heavy chain comprising the amino acid sequence of SEQ ID NO: 41, and a light chain comprising the amino acid sequence of SEQ ID NO: 42.

19. The method of claim 1, wherein the first antigen-binding arm of the bispecific antibody comprises three heavy chain CDRs (A-HCDR1, A-HCDR2 and A-HCDR3) of a heavy chain variable region (A-HCVR) comprising the amino acid sequence of SEQ ID NO: 1 and three light chain CDRs (A-LCDR1, A-LCDR2 and A-LCDR3) of a light chain variable region (A-LCVR) comprising the amino acid sequence of SEQ ID NO: 2.

20. The method of claim 19, wherein A-HCDR1 comprises the amino acid sequence of SEQ ID NO: 8; A-HCDR2 comprises the amino acid sequence of SEQ ID NO: 9; A-HCDR3 comprises the amino acid sequence of SEQ ID NO: 10; A-LCDR1 comprises the amino acid sequence of SEQ ID NO: 11; A-LCDR2 comprises the amino acid sequence of SEQ ID NO: 12; and A-LCDR3 comprises the amino acid sequence of SEQ ID NO: 13.

21. The method of claim 20, wherein the bispecific antibody comprises a A-HCVR comprising the amino acid sequence of SEQ ID NO:1 and a A-LCVR comprising the amino acid sequence of SEQ ID NO:2.

22. The method of claim 1, wherein the second antigen-binding arm of the bispecific antibody comprises three heavy chain CDRs (B-HCDR1, B-HCDR2 and B-HCDR3) of a heavy chain variable region (B-HCVR) comprising an amino acid sequence selected from the group consisting of SEQ ID NOs: 3, 4, 5, 6 and 7, and three light chain CDRs (B-LCDR1, B-LCDR2 and B-LCDR3) of a light chain variable region (B-LCVR) comprising the amino acid sequence of SEQ ID NO: 2.

23. The method of claim 22, wherein B-HCDR1, B-HCDR2 and B-HCDR3 comprise, respectively, the amino acid sequences selected from the group consisting of SEQ ID NOs: 14-15-16, 17-18-19, 20-21-22, 23-24-25, and 26-27-28; and B-LCDR1, B-LCDR2 and B-LCDR3 comprise, respectively, the amino acid sequences of SEQ ID NOs: 11-12-13.

24. The method of claim 23, wherein the bispecific antibody comprises a B-HCVR comprising an amino acid sequence selected from the group consisting of SEQ ID NOs: 3, 4, 5, 6 and 7, and a B-LCVR comprising the amino acid sequence of SEQ ID NO: 2.

25. The method of claim 22, wherein the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3, and three light chain CDRs (B-LCDR1, B-LCDR2 and B-LCDR3) of a light chain variable region (B-LCVR) comprising the amino acid sequence of SEQ ID NO: 2.

26. The method of claim 22, wherein the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 4, and three light chain CDRs (B-LCDR1, B-LCDR2 and B-LCDR3) of a light chain variable region (B-LCVR) comprising the amino acid sequence of SEQ ID NO: 2.

27. The method of claim 22, wherein the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 5, and three light chain CDRs (B-LCDR1, B-LCDR2 and B-LCDR3) of a light chain variable region (B-LCVR) comprising the amino acid sequence of SEQ ID NO: 2.

28. The method of claim 22, wherein the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 6, and three light chain CDRs (B-LCDR1, B-LCDR2 and B-LCDR3) of a light chain variable region (B-LCVR) comprising the amino acid sequence of SEQ ID NO: 2.

29. The method of claim 22, wherein the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 7, and three light chain CDRs (B-LCDR1, B-LCDR2 and B-LCDR3) of a light chain variable region (B-LCVR) comprising the amino acid sequence of SEQ ID NO: 2.

30. The method of claim 1, wherein the anti-CTLA-4 antibody, the bispecific antibody, or both, comprise a human IgG1 or IgG4 heavy chain constant region.

31. A method of treating or inhibiting the growth of a MUC16-expressing tumor comprising administering to a subject in need thereof (a) a therapeutically effective amount of an antibody or antigen-binding fragment thereof that specifically binds cytotoxic T-lymphocyte-associated protein 4 (CTLA-4); and (b) a therapeutically effective amount of a bispecific antibody comprising a first antigen-binding arm that specifically binds MUC16 and a second antigen-binding arm that specifically binds CD3, wherein:
(a) the anti-CTLA-4 antibody or antigen-binding fragment thereof comprises three heavy chain complementarity determining regions (CDRs) (HCDR1, HCDR2 and HCDR3) of a heavy chain variable region (HCVR) comprising the amino acid sequence of SEQ ID NO: 33, and three light chain complementarity determining regions (LCDR1, LCDR2 and LCDR3) of a light chain variable region (LCVR) comprising the amino acid sequence of SEQ ID NO: 34;
(b) the first antigen-binding arm of the bispecific antibody comprises three heavy chain CDRs (A-HCDR1, A-HCDR2 and A-HCDR3) of a heavy chain variable region (A-HCVR) comprising the amino acid sequence of SEQ ID NO: 1 and three light chain CDRs (A-LCDR1, A-LCDR2 and A-LCDR3) of a light chain variable region (A-LCVR) comprising the amino acid sequence of SEQ ID NO: 2; and
(c) the second antigen-binding arm of the bispecific antibody comprises three heavy chain CDRs (B-HCDR1, B-HCDR2 and B-HCDR3) of a heavy chain variable region (B-HCVR) comprising the amino acid sequence of SEQ ID NO: 3, and three light chain CDRs (B-LCDR1, B-LCDR2 and B-LCDR3) of a light chain variable region (B-LCVR) comprising the amino acid sequence of SEQ ID NO: 2.

32. The method of claim 31, wherein:
(a) HCDR1 comprises the amino acid sequence of SEQ ID NO: 35; HCDR2 comprises the amino acid sequence of SEQ ID NO: 36; HCDR3 comprises the amino acid sequence of SEQ ID NO: 37; LCDR1 comprises the amino acid sequence of SEQ ID NO: 38; LCDR2 comprises the amino acid sequence of SEQ ID NO: 39; and LCDR3 comprises the amino acid sequence of SEQ ID NO: 40;
(b) A-HCDR1 comprises the amino acid sequence of SEQ ID NO: 8; A-HCDR2 comprises the amino acid sequence of SEQ ID NO: 9; A-HCDR3 comprises the amino acid sequence of SEQ ID NO: 10; A-LCDR1 comprises the amino acid sequence of SEQ ID NO: 11; A-LCDR2 comprises the amino acid sequence of SEQ ID NO: 12; and A-LCDR3 comprises the amino acid sequence of SEQ ID NO: 13; and
(c) B-HCDR1 comprises the amino acid sequence of SEQ ID NO: 14; B-HCDR2 comprises the amino acid sequence of SEQ ID NO: 15; B-HCDR3 comprises the amino acid sequence of SEQ ID NO: 16; B-LCDR1 comprises the amino acid sequence of SEQ ID NO: 11; B-LCDR2 comprises the amino acid sequence of SEQ ID NO: 12; and B-LCDR3 comprises the amino acid sequence of SEQ ID NO: 13.

33. The method of claim 32, wherein:
(a) the anti-CTLA4 antibody or antigen-binding fragment thereof comprises a HCVR comprising the amino acid sequence of SEQ ID NO: 33, and the LCVR comprising the amino acid sequence of SEQ ID NO: 34;
(b) the bispecific antibody comprises a A-HCVR comprising the amino acid sequence of SEQ ID NO:1 and the A-LCVR comprising the amino acid sequence of SEQ ID NO:2; and
(c) the bispecific antibody comprises a B-HCVR comprising the amino acid sequence of SEQ ID NO: 3, and the B-LCVR comprising the amino acid sequence of SEQ ID NO: 2.

34. The method of claim 33, wherein:
(a) the anti-CTLA-4 antibody comprises a heavy chain comprising the amino acid sequence of SEQ ID NO: 41, and a light chain comprising the amino acid sequence of SEQ ID NO: 42;
(b) the first antigen binding arm of the bispecific antibody comprises a heavy chain comprising the amino acid sequence of SEQ ID NO: 29, and a light chain comprising the amino acid sequence of SEQ ID NO: 30; and
(c) the second antigen binding arm of the bispecific antibody comprises a heavy chain comprising the amino acid sequence of SEQ ID NO: 31, and a light chain comprising the amino acid sequence of SEQ ID NO: 30.

35. The method of claim 31, wherein the tumor comprises an ovarian cancer.

36. The method of claim 1, wherein administration of the anti-CTLA4 antibody or antigen-binding fragment thereof and the bispecific antibody results in an increase in serum cytokine levels in the subject of no more than 5% relative to an isotype control at four hours post-administration.
